(19) United States
(12) Patent Application Publication
(10) Pub. No.: US 2003/0045757 A1
(43) Pub. Date: Mar. 6, 2003

(54) Modified polyphenylene ether

(76) Inventors: Yoshiyuki Ishii, Shizuoka-ken (JP); Kumi Sorita, Tokyo (JP)

Correspondence Address: BIRCH STEWART KOLASCH & BIRCH, PO BOX 747, FALLS CHURCH, VA, 22040-0747, US

(21) Appl. No.: 10/182,648

(22) PCT Filed: Feb. 23, 2001
(86) PCT No.: PCT/JP01/01358

(30) Foreign Application Priority Data
Feb. 23, 2000 (JP) 2000-45418
Mar. 22, 2000 (JP) 2000-79660

(51) Int. Cl. C08G 63/00; C08G 65/38; C07C 43/263

(52) U.S. Cl. 568/660; 528/219; 528/176

(57) ABSTRACT

Disclosed is a modified polyphenylene ether having a number average molecular weight of not smaller than 4,000, which is obtained by reacting a precursory modified polyphenylene ether with a phenolic compound in the presence of a radical generator, wherein the precursory modified polyphenylene ether comprises a plurality of polyphenylene ether chains each having a terminal phenolic hydroxyl group, wherein the plurality of polyphenylene ether chains collectively have at least one terminal phenolic hydroxyl group modified and wherein not all of the terminal phenolic hydroxyl groups of the plurality of polyphenylene ether chains are modified. Also disclosed is a method for producing the modified polyphenylene ether.

### BACKGROUND OF THE INVENTION

1. Field of the Invention

The present invention relates to a modified polyphenylene ether. More particularly, the present invention is concerned with a modified polyphenylene ether having a number average molecular weight of not smaller than 4,000, which is obtained by reacting a precursory modified polyphenylene ether with a phenolic compound in the presence of a radical generator, wherein the precursory modified polyphenylene ether comprises a plurality of polyphenylene ether chains each having a terminal phenolic hydroxyl group, and wherein a part of the plurality of polyphenylene ether chains are modified. The present invention is also concerned with a method for producing the modified polyphenylene ether.

The modified polyphenylene ether of the present invention exhibits excellent melt properties, excellent mechanical properties and excellent film-forming properties, so that the modified polyphenylene ether can be advantageously used in a curable film and a multi-layer circuit board as well as in the build-up method and the like.

2. Prior Art

Conventionally, as a circuit board (e.g., a multilayer circuit board used for packaging a semiconductor element), a multi-layer printed circuit board is known. A multi-layer printed circuit board is usually obtained as follows. A copper foil is attached onto an insulating substrate comprising a resin. The copper foil is subjected to etching to form a circuit in the copper foil, thereby obtaining a copper-clad substrate having a circuit in the copper foil. Then, a plurality of such copper-clad substrates are disposed one upon another to thereby obtain a multi-layer printed circuit board.

In the production of such a conventional multilayer printed circuit board, through-holes are formed through the copper-clad substrates, and plating or the like is performed on the inside surfaces of the through-holes so as to cause the copper-clad substrates to be electrically connected with each other. The necessity of the formation of the through-holes poses a problem in that the circuit design of the printed circuit board is inevitably limited, making it difficult to achieve a high density wiring of the printed circuit board. Therefore, the conventional multi-layer printed circuit board cannot satisfy the recent strong demands for an improved multi-layer printed circuit board which has a wiring having a higher precision and a higher density.

In order to solve such problem, a new method (which is called the “build-up method”) for producing a multi-layer printed circuit board has been developed. In the build-up method, insulating layers and circuit layers are alternately formed upon the surface of a substrate. The method involves coating, plating, and formation of via holes and the like, to thereby obtain a multi-layer printed circuit board.

Examples of build-up methods include a subtractive method, in which a copper foil having laminated thereon a curable resin film is laminated on a circuit substrate, and a circuit is formed in the surface of the copper foil by etching; and an additive method, in which a curable resin film (which is not laminated on a copper foil) is laminated on a circuit substrate, the resin film is cured, and the cured resin film is subjected to electroless plating so as to form a circuit on the cured resin film.

In the build-up method, it is required that, in the lamination step, a resin used for forming an insulating layer flow over a circuit board so as to form a complete resin layer having no void therein. It is also required that the resin used for forming an insulating layer have an elasticity satisfactory for enduring a subsequent processing step. Therefore, as a resin for forming an insulating layer in a circuit board, thermocurable resins (especially, an epoxy resin) have conventionally been used for many years.

However, due to the great improvements in the performance of electronic equipment in recent years, as a material for an insulating layer in a circuit board, an epoxy resin has become unsatisfactory from the view-point of electric characteristics, heat resistance and water absorption resistance.

For solving this problem, polyphenylene ethers and modified polyphenylene ethers have recently been attracting attention as new materials for an insulating layer in a circuit board. Especially, modified polyphenylene ethers are drawing great attention for the reason that modified polyphenylene ethers exhibit excellent film-forming properties and excellent adhesion to a metal layer.

However, a conventional polyphenylene ether and a conventional modified polyphenylene ether, each of which is a thermoplastic resin, have a problem in that when either of them is incorporated in a thermocurable resin to obtain a thermocurable resin composition, the melt viscosity of the thermocurable resin composition becomes much higher than that of the thermocurable resin, so that not only is a high pressure needed for shaping the thermocurable resin composition, but also it becomes difficult to fill the interstices of a fine circuit structure with the thermocurable resin composition.

Therefore, it has been desired to develop a polyphenylene ether or a modified polyphenylene ether which has excellent melt properties and has an advantage in that a thermocurable resin composition containing the polyphenylene ether or modified polyphenylene ether can be easily filled in the interstices of a fine circuit structure.

In general, the melt properties of a polyphenylene ether can be improved by lowering the melt viscosity of the polyphenylene ether. As a method for lowering the melt viscosity of a polyphenylene ether, there can be mentioned a method comprising lowering the number average molecular weight of a polyphenylene ether. For example, Unexamined Japanese Patent Application Laid-Open Specification No. Hei 11-236430 (corresponding to EP 921158 A2) discloses a method which comprises performing a redistribution reaction of a polyphenylene ether with a phenolic compound in the presence of a peroxide to thereby lower the number average molecular weight of the polyphenylene ether. In this method, in accordance with the decrease in the number average molecular weight of the polyphenylene ether, the melt viscosity of the polyphenylene ether is lowered. However, this method is disadvantageous in that the polyphenylene ether obtained by the redistribution reaction has substantially no high molecular weight component remaining therein, so that the polyphenylene ether exhibits extremely poor mechanical properties and extremely poor film-forming properties. As a result, it becomes impossible to produce a good film from the polyphenylene ether obtained by this method. Needless to say, a film produced from the polyphenylene ether obtained by this method cannot be used in the build-up method.

Unexamined Japanese Patent Application Laid-Open Specification No. Hei 11-302529 (corresponding to U.S. Pat. No. 5,834,565) discloses a resin composition for use in a printed circuit board, wherein the resin composition comprises a thermocurable resin and a polyphenylene ether having a number average molecular weight of less than 3,000. The above-mentioned patent document discloses a method for producing a polyphenylene ether having a number average molecular weight of less than 3,000, wherein the method comprises performing a redistribution reaction of a polyphenylene ether with a phenolic compound in the presence of a peroxide to thereby lower the number average molecular weight of the polyphenylene ether. The polyphenylene ether obtained by this method, which has a number average molecular weight of less than 3,000, is improved in the melt processing properties. However, the polyphenylene ether has an unsatisfactory mechanical strength. Further, the polyphenylene ether exhibits unsatisfactory film-forming properties, and, therefore, when the polyphenylene ether is dissolved in a solvent and the resultant solution is used for forming a cast film, it is impossible to obtain a practical film. (It is noted that although the above-mentioned Unexamined Japanese Patent Application Laid-Open Specification No. Hei 11-302529 states that a modified polyphenylene ether can also be used, no modified polyphenylene ether is used in the working examples of the above-mentioned patent document.)

In order to solve such problems, Unexamined Japanese Patent Application Laid-Open Specification No. Hei 7-278293 discloses a method for improving the properties of a polyphenylene ether, which comprises contacting a polyphenylene ether with a quinone in the presence of a secondary amine under conditions wherein the temperature is in the range of from 50 to 120° C. and the polyphenylene ether is not completely dissolved. However, since this method uses a secondary amine as a catalyst, the removal of the secondary amine is needed when the polyphenylene ether which has been treated by this method is used for forming a film for use in the above-mentioned build-up method or the like. The above-mentioned patent document has no description about a modified polyphenylene ether.

Unexamined Japanese Patent Application Laid-Open Specification No. Hei 10-204173 discloses a method for improving the properties of a polyphenylene ether. This method is as follows. A phenolic monomer is subjected to an oxidative polymerization using a complex of a copper compound and at least one amine as a catalyst in the presence of a secondary amine while feeding oxygen to the reaction system to thereby obtain a reaction mixture containing a polyphenylene ether. A part of the polyphenylene ether in the reaction mixture is caused to be deposited during or after the polymerization, to thereby obtain a slurry. The slurry is subjected to heat treatment at a temperature which is in the range of from 50 to 120° C. but at which the polyphenylene ether is not completely dissolved, while stopping the feeding of oxygen, to thereby improve the properties of the polyphenylene ether. However, the removal of the copper compound and amine used as catalysts is needed when the polyphenylene ether which has been treated by this method is used for forming a film for use in the above-mentioned build-up method or the like. The above-mentioned patent document has no description about a modified polyphenylene ether.

As can be seen from the above, by the conventional methods, it is impossible to provide a modified polyphenylene ether which exhibits excellent melt properties, excellent mechanical properties and excellent film-forming properties.

### SUMMARY OF THE INVENTION

In this situation, the present inventors have made extensive and intensive studies with a view toward developing a modified polyphenylene ether which exhibits excellent melt properties, excellent mechanical properties and excellent film-forming properties and hence which can be advantageously used in a curable film and a multi-layer circuit board as well as in the build-up method and the like.

As a result, it has unexpectedly been found that the above-mentioned excellent properties can be exhibited by a modified polyphenylene ether having a number average molecular weight of not smaller than 4,000, which is obtained by reacting a precursory modified polyphenylene ether with a phenolic compound in the presence of a radical generator, wherein the precursory modified polyphenylene ether comprises a plurality of polyphenylene ether chains each having a terminal phenolic hydroxyl group, wherein the plurality of polyphenylene ether chains collectively have at least one terminal phenolic hydroxyl group modified and wherein, however, not all of the terminal phenolic hydroxyl groups of the plurality of polyphenylene ether chains are modified. This modified polyphenylene ether can be advantageously used in a curable film and a multi-layer circuit board as well as in the build-up method and the like. Based on this finding, the present invention has been completed.

Accordingly, it is a primary object of the present invention to provide a modified polyphenylene ether which exhibits excellent melt properties, excellent mechanical properties and excellent film-forming property.

It is another object of the present invention to provide a method for producing the above-mentioned modified polyphenylene ether.

The foregoing and other objects, features and advantages of the present invention will be apparent from the following detailed description and appended claims taken in connection with the accompanying drawings.

### BRIEF DESCRIPTION OF THE DRAWINGS

In the drawings:

FIG. 1 shows a graph obtained by plotting the ratio of change of the number average molecular weight (Mn) of the polyphenylene ether (PPE) against the amount of bisphenol A, wherein the graph is based on the results of Examples 1 to 3 and Comparative Examples 1 to 3;

FIG. 2 shows a graph obtained by plotting the ratio of change of the weight average molecular weight (Mw) of the PPE against the amount of bisphenol A, wherein the graph is based on the results of Examples 1 to 3 and Comparative Examples 1 to 3; and

FIG. 3 shows a graph obtained by plotting the ratio of change of the Z-average molecular weight (Mz) of the PPE against the amount of bisphenol A, wherein the graph is based on the results of Examples 1 to 3 and Comparative Examples 1 to 3.

### DETAILED DESCRIPTION OF THE INVENTION

In one aspect of the present invention, there is provided a modified polyphenylene ether having a number average molecular weight of not smaller than 4,000, which is obtained by reacting a precursory modified polyphenylene ether with a phenolic compound in the presence of a radical generator, the precursory modified polyphenylene ether comprising a plurality of polyphenylene ether chains each having a terminal phenolic hydroxyl group, the plurality of polyphenylene ether chains collectively having at least one terminal phenolic hydroxyl group modified, wherein not all of the terminal phenolic hydroxyl groups of the plurality of polyphenylene ether chains are modified.

In another aspect of the present invention, there is provided a method for producing a modified polyphenylene ether having a number average molecular weight of not smaller than 4,000, which comprises performing a homogeneous reaction of a precursory modified polyphenylene ether with a phenolic compound in the presence of a radical generator,

the precursory modified polyphenylene ether comprising a plurality of polyphenylene ether chains each having a terminal phenolic hydroxyl group, the plurality of polyphenylene ether chains collectively having at least one terminal phenolic hydroxyl group modified, wherein not all of the terminal phenolic hydroxyl groups of the plurality of polyphenylene ether chains are modified.

For easy understanding of the present invention, the essential features and various preferred embodiments of the present invention are enumerated below.

1. A modified polyphenylene ether having a number average molecular weight of not smaller than 4,000, which is obtained by reacting a precursory modified polyphenylene ether with a phenolic compound in the presence of a radical generator,

the precursory modified polyphenylene ether comprising a plurality of polyphenylene ether chains each having a terminal phenolic hydroxyl group, the plurality of polyphenylene ether chains collectively having at least one terminal phenolic hydroxyl group modified, wherein not all of the terminal phenolic hydroxyl groups of the plurality of polyphenylene ether chains are modified.

2. The modified polyphenylene ether according to item 1 above, wherein the precursory modified polyphenylene ether is a reaction product of a polyphenylene ether with an unsaturated carboxylic acid or an unsaturated carboxylic anhydride.

3. The modified polyphenylene ether according to item 1 above, wherein the precursory modified polyphenylene ether is a reaction product of a polyphenylene ether with at least one vinyl compound selected from the group consisting of an acrylic ester and a methacrylic ester, each of the acrylic ester and the methacrylic ester containing a C₉-C₂₂ alkyl group, a C₉-C₂₂ alkenyl group, a C₉-C₂₂ aralkyl group or a C₉-C₂₂ cycloalkyl group.

4. A curable polyphenylene ether resin composition comprising 1 to 99 parts by weight of the modified polyphenylene ether of any one of items 1 to 3 above and 99 to 1 part by weight of a thermocurable resin, the total amount of the modified polyphenylene ether and the thermocurable resin being 100 parts by weight.

5. The curable polyphenylene ether resin composition according to item 4 above, wherein the thermocurable resin is at least one member selected from the group consisting of triallyl isocyanurate, triallyl cyanurate, a cyanate ester resin, an epoxy resin and a benzoxazine resin.

6. A curable film comprising the curable polyphenylene ether resin composition of item 4 or 5 above.

7. A cured film obtained by curing the curable film of item 6 above.

8. A curable structure comprising an insulating layer of the curable film of item 6 above and, disposed thereon, an electroconductive layer of a metallic foil.

9. A cured structure obtained by curing the curable structure of item 8 above.

10. A curable composite material comprising a substrate and the curable polyphenylene ether resin composition of item 4 or 5 above.

11. A cured composite material obtained by curing the curable composite material of item 10 above.

12. A method for producing a modified polyphenylene ether having a number average molecular weight of not smaller than 4,000, which comprises performing a homogeneous reaction of a precursory modified polyphenylene ether with a phenolic compound in the presence of a radical generator,

the precursory modified polyphenylene ether comprising a plurality of polyphenylene ether chains each having a terminal phenolic hydroxyl group, the plurality of polyphenylene ether chains collectively having at least one terminal phenolic hydroxyl group modified, wherein not all of the terminal phenolic hydroxyl groups of the plurality of polyphenylene ether chains are modified.

13. A method for producing a varnish of a curable polyphenylene ether resin composition, which comprises:

performing a homogeneous reaction of a precursory modified polyphenylene ether with a phenolic compound in the presence of a radical generator,

the precursory modified polyphenylene ether comprising a plurality of polyphenylene ether chains each having a terminal phenolic hydroxyl group, the plurality of polyphenylene ether chains collectively having at least one terminal phenolic hydroxyl group modified, wherein not all of the terminal phenolic hydroxyl groups of the plurality of polyphenylene ether chains are modified,

thereby obtaining a reaction mixture containing a modified polyphenylene ether having a number average molecular weight of not smaller than 4,000,

isolating the modified polyphenylene ether from the reaction mixture, and

adding the isolated, modified polyphenylene ether and a thermocurable resin to an organic solvent, followed by mixing, to thereby obtain a curable polyphenylene ether resin composition in a varnish form.

14. A method for producing a varnish of a curable polyphenylene ether resin composition, which comprises:

performing a homogeneous reaction of a precursory modified polyphenylene ether with a phenolic compound in the presence of an organic solvent and a radical generator,

the precursory modified polyphenylene ether comprising a plurality of polyphenylene ether chains each having a terminal phenolic hydroxyl group, the plurality of polyphenylene ether chains collectively having at least one terminal phenolic hydroxyl group modified, wherein not all of the terminal phenolic hydroxyl groups of the plurality of polyphenylene ether chains are modified,

thereby obtaining a reaction mixture containing a modified polyphenylene ether having a number average molecular weight of not smaller than 4,000, and

adding a thermocurable resin to the reaction mixture to obtain a curable polyphenylene ether resin composition in a varnish form.

15. The method according to item 13 or 14 above, wherein the curable polyphenylene ether resin composition comprises 1 to 99 parts by weight of the modified polyphenylene ether and 99 to 1 part by weight of the thermocurable resin, the total amount of the modified polyphenylene ether and the thermocurable resin being 100 parts by weight.

16. The method according to item 15 above, wherein the thermocurable resin is at least one member selected from the group consisting of triallyl isocyanurate, triallyl cyanurate, a cyanate ester resin, an epoxy resin and a benzoxazine resin.

17. A method for producing a curable film, which comprises applying a varnish obtained by the method of any one of items 13 to 16 above onto a support to obtain a coating, and peeling off the coating from the support to obtain a curable film.

Hereinbelow, the present invention is described in detail.

The modified polyphenylene ether of the present invention has a number average molecular weight of not smaller than 4,000. It is preferred that the number average molecular weight of the modified polyphenylene ether is from 4,000 to 30,000, more advantageously from 4,000 to 20,000, still more advantageously from 5,000 to 20,000. When the number average molecular weight of the modified polyphenylene ether is smaller than 4,000, the mechanical properties of a film or the like produced using the modified polyphenylene ether become poor.

The broadness of the molecular weight distribution of the modified polyphenylene ether can be expressed by the polydispersity (Mw/Mn) thereof, which is a value obtained by dividing the weight average molecular weight (Mw) by the number average molecular weight (Mn). It is preferred that the polydispersity (Mw/Mn) of the modified polyphenylene ether is from 2.0 to 6.0, more advantageously from 2.1 to 5.0, still more advantageously from 2.2 to 4.0.

When the polydispersity is smaller than 2.0, it tends to become difficult to achieve a good balance between the melt properties of the modified polyphenylene ether and the mechanical properties of a shaped article produced using the modified polyphenylene ether. On the other hand, when the polydispersity is larger than 6.0, the modified polyphenylene ether has too large a content of high molecular weight components, so that it becomes difficult to lower the melt viscosity of the modified polyphenylene ether.

The modified polyphenylene ether of the present invention is obtained by reacting a precursory modified polyphenylene ether with a phenolic compound in the presence of a radical generator, wherein the precursory modified polyphenylene ether comprises a plurality of polyphenylene ether chains each having a terminal phenolic hydroxyl group, wherein the plurality of polyphenylene ether chains collectively have at least one terminal phenolic hydroxyl group modified and wherein, however, not all of the terminal phenolic hydroxyl groups of the plurality of polyphenylene ether chains are modified.

With respect to the terminal phenolic hydroxyl groups of the plurality of polyphenylene ether chains of the precursory modified polyphenylene ether, the number of terminal phenolic hydroxyl groups modified is generally from 0.01 to less than 90, preferably from 1 to less than 80, relative to 100 terminal phenolic hydroxyl groups.

When the above-mentioned number of terminal phenolic hydroxyl groups modified in the precursory modified polyphenylene ether is smaller than 0.01, the modified polyphenylene ether has too low a content of high molecular weight components, so that it is likely that the mechanical properties of the modified polyphenylene ether become poor and, hence, the mechanical properties of a shaped article (e.g., a curable film) obtained using the modified polyphenylene ether become poor.

On the other hand, when the above-mentioned number of terminal phenolic hydroxyl groups modified in the precursory modified polyphenylene ether is 90 or more, there is a tendency that the number average molecular weight of the modified polyphenylene ether cannot be satisfactorily lowered, so that the melt properties of the modified polyphenylene ether cannot be improved and, hence, the melt properties of a resin composition containing the modified polyphenylene ether cannot be improved.

The above-mentioned number of terminal phenolic hydroxyl groups modified in the precursory modified polyphenylene, relative to 100 terminal phenolic hydroxyl groups, can be measured by means of a nuclear magnetic resonance apparatus, an infrared spectrometer or the like.

The number average molecular weight of the precursory modified polyphenylene ether is preferably from 5,000 to 50,000, more preferably from 10,000 to 30,000. When the number average molecular weight of the precursory modified polyphenylene ether is more than 50,000, there is a tendency that the melt flow properties of the precursory modified polyphenylene ether are largely influenced by the melt flow properties of the high molecular weight components thereof, so that it becomes difficult to improve the melt flow properties of the precursory modified polyphenylene ether.

As an example of a radical generator used in the reaction of the precursory modified polyphenylene ether with the phenolic compound, there can be mentioned a peroxide represented by the following formula:

Z¹—O—O—Z²

wherein each of Z¹ and Z² independently represents a hydrogen atom, an alkyl group, an aryl group, an aroyl group, an alkanoyl group, an alkenoyl group, an alkoxycarbonyl group, a sulfuryl group, a sulfonyl group or a phosphoryl group.

Specific examples of peroxides represented by the above-mentioned formula include benzoyl peroxide; benzoyl peroxide derivatives, such as di(4-butyl benzoyl) peroxide; dilauryl peroxide; diacyl peroxides, such as acetylbenzoyl peroxide, acetylcyclohexylsulfonyl peroxide and diphthaloyl peroxide; peroxy dicarbonates, such as diacetylperoxy dicarbonate, di-n-propylperoxy dicarbonate, diisopropyl peroxide and bis(4-t-butylcyclohexyl)peroxy dicarbonate; perbenzoic acid; perbenzoic acid derivatives, such as 3-chloroperbenzoic acid and 4-nitroperbenzoic acid; peroxycarboxylic acids, such as peroxyacetic acid, peroxycarboxylic acid, peroxybutanoic acid, peroxynonanoic acid, peroxydodecanoic acid, diperoxyglutaric acid, diperoxyadipic acid, diperoxyoctanedioic acid, diperoxynonanedioic acid, diperoxydodecanedioic acid and monoperoxyphthalic acid; inorganic peroxo acids, such as peroxomonosulfuric acid, peroxodisulfuric acid, peroxomonophosphoric acid and peroxodiphosphoric acid; salts of these inorganic peroxo acids; and peroxycarboxylic acid esters, such as t-butyl performate, t-butyl peracetate, t-butyl peroxyisobutyrate, t-butyl peroxynonanoate, t-butyl monoperoxymaleate, t-butyl diperoxyphthalate, di-t-butyl diperoxyadipate and 2,5-dimethyl-2,5-bis(benzoylperoxy)hexane.

Further examples of radical generators include quinones and diphenoquinones, such as benzoquinone and 2,2′-6,6′-tetramethyldiphenoquinone (TMDQ).

The amount of the radical generator is preferably from 0.1 to 20 parts by weight, more preferably from 0.5 to 10 parts by weight, relative to 100 parts by weight of the precursory modified polyphenylene ether.

When the amount of the radical generator is less than 0.1 part by weight, there is a tendency that the below-mentioned redistribution reaction does not easily advance, so that it becomes difficult to obtain a modified polyphenylene ether having a desired number average molecular weight. On the other hand, when the amount of the radical generator is more than 20 parts by weight, there is a tendency that the redistribution reaction advances too vigorously, making it difficult to control the redistribution.

The precursory modified polyphenylene ether used in the present invention can be obtained by modifying a polyphenylene ether represented by the following formula (1):

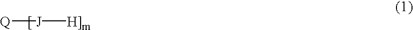

wherein:

m is an integer of from 1 to 6,

J represents a polyphenylene ether chain consisting essentially of recurring units each represented by the following formula (2):

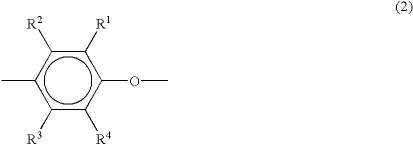

wherein each of R¹, R², R³ and R⁴ independently represents a lower alkyl group, an aryl group, a haloalkyl group, a halogen atom or a hydrogen atom, and

when m is 1, Q represents a hydrogen atom, and when m is 2 or more, Q represents a residue of a multi-functional phenolic compound having 2 to 6 phenolic hydroxyl groups in the molecule and having unpolymerizable substituents at the ortho and para positions with respect to the phenolic hydroxyl groups.

As specific examples of R¹, R², R³ and R⁴ in formula (2) above, there can be mentioned the following groups or atoms. Specific examples of lower alkyl groups include a methyl group, an ethyl group, an n-propyl group, an isopropyl group, an n-butyl group and an isobutyl group. A specific example of an aryl group is a phenyl group. Specific examples of halogen atoms include a bromine atom and a chlorine atom. Specific examples of haloalkyl groups include a bromomethyl group and a chloromethyl group.

Representative examples of Q in formula (1) above wherein m is 2 or more include residues represented by the four types of the following formulae (3):

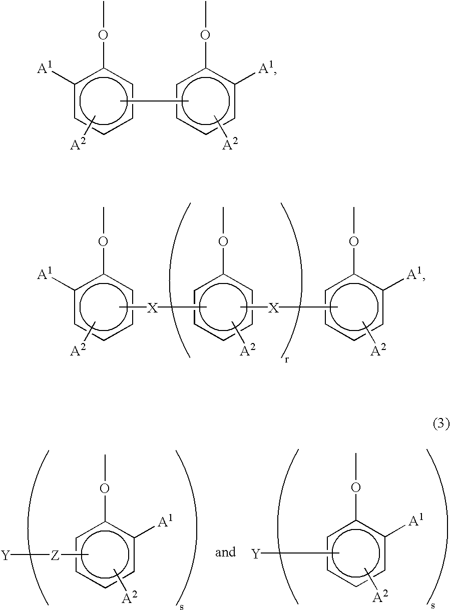

wherein:

each of A¹ and A² independently represents a straight chain alkyl group having 1 to 4 carbon atoms,

each X independently represents an aliphatic hydrocarbon residue or a substitution derivative thereof; an aralkyl group or a substitution derivative thereof; an oxygen atom; a sulfur atom; a sulfonyl group; or a carbonyl group,

Y represents an aliphatic hydrocarbon residue or a substitution derivative thereof; an aromatic hydrocarbon residue or a substitution derivative thereof; or an aralkyl group or a substitution derivative thereof,

each Z independently represents an oxygen atom, a sulfur atom, a sulfonyl group or a carbonyl group,

r is an integer of from 0 to 4, and

s is an integer of from 2 to 6, with the proviso that:

each of A², X and Z is bonded directly to and Y is bonded directly or indirectly to the ortho or para position with respect to the oxygen atom shown in the formulae above, and

when directly to a first substituted phenoxy group is bonded the phenyl moiety of a second substituted phenoxy group, the phenyl moiety is bonded to the ortho or para position with respect to the oxygen atom shown in the formulae above. Specific examples of Q include residues represented by the following formulae (4) and (5):

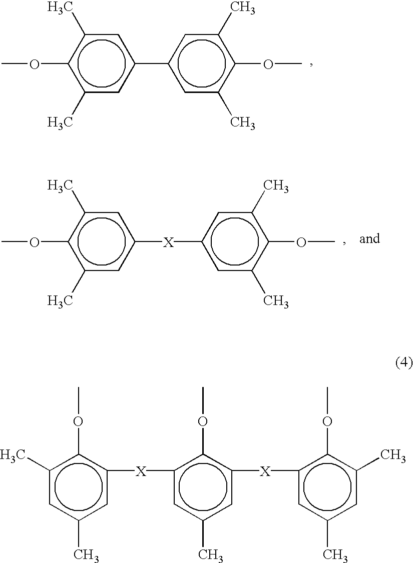

wherein each X independently represents —CH₂—, —C(CH₃)₂—, —O—, —S—, —SO₂—, or —CO—, and

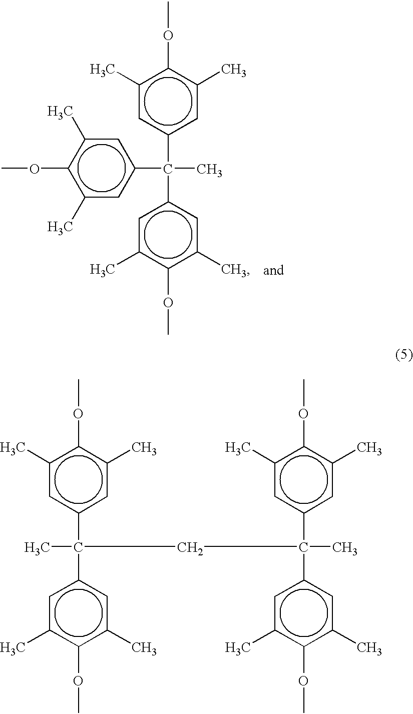

A polyphenylene ether chain represented by J in formula (1) above may, in addition to a unit represented by formula (2) above, contain a unit represented by the following formula (6):

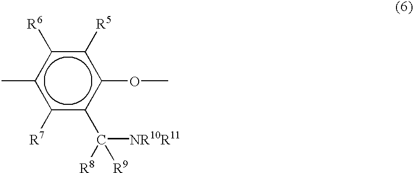

wherein:

each of R⁵, R⁶, R⁷, R⁸ and R⁹ independently represents a hydrogen atom, a halogen atom, a lower alkyl group, an aryl group, or a haloalkyl group, and

each of R¹⁰ and R¹¹ independently represents a hydrogen atom; an alkyl group having 1 to 10 (preferably 1 to 5) carbon atoms which is unsubstituted or substituted with an aryl group, a halogen atom or the like; or an aryl group which is unsubstituted or substituted with an aryl group, a halogen atom or the like, with the proviso that R¹⁰ and R¹l are not simultaneously hydrogen atoms.

Specific examples of units represented by formula (6) above include a unit represented by the following formula (7):

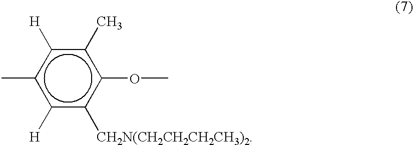

In the present invention, preferred examples of polyphenylene ethers represented by formula (1) above include: a poly(2,6-dimethyl-1,4-phenylene ether) obtained by homopolymerizing 2,6-dimethylphenol; a styrene-grafted copolymer of the poly(2,6-dimethyl-1,4-phenylene ether); a copolymer of 2,6-dimethylphenol and 2,3,6-trimethylphenol; a copolymer of 2,6-dimethylphenol and 2,6-dimethyl-3-phenylphenol; and a multi-functional polyphenylene ether obtained by polymerizing 2,6-dimethylphenol in the presence of a multifunctional phenolic compound represented by the following formula (8):

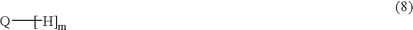

wherein m is an integer of from 2 to 6 and Q represents a residue of a multi-functional phenolic compound having 2 to 6 phenolic hydroxyl groups in the molecule and having unpolymerizable substituents at the ortho and para positions with respect to the phenolic hydroxyl groups.

As described above, the precursory modified polyphenylene ether used in the present invention comprises a plurality of polyphenylene ether chains each having a terminal phenolic hydroxyl group, wherein the plurality of polyphenylene ether chains collectively have at least one terminal phenolic hydroxyl group modified and wherein, however, not all of the terminal phenolic hydroxyl groups of the plurality of polyphenylene ether chains are modified.

In the present invention, it is preferred that the precursory modified polyphenylene ether is a product of the reaction of a polyphenylene ether represented by formula (1) above with an unsaturated carboxylic acid or an unsaturated carboxylic anhydride.

In the present invention, it is also preferred that the precursory modified polyphenylene ether is a product of the reaction of a polyphenylene ether represented by formula (1) above with at least one vinyl compound selected from the group consisting of an acrylic ester and a methacrylic ester.

Examples of unsaturated carboxylic acids and unsaturated carboxylic anhydrides include acrylic acid, methacrylic acid, maleic anhydride, fumaric acid, itaconic acid, itaconic anhydride, glutaconic anhydride and citraconic anhydride. Of these compounds, maleic anhydride and fumaric acid are especially preferred.

With respect to the structure of a maleic anhydride-modified polyphenylene ether, reference can be made to J. H. Glans and Akkapeddi, “Macromolecules”, vol. 24, pp. 383-386 (1991).

The reaction of a polyphenylene ether with an unsaturated carboxylic acid or unsaturated carboxylic anhydride is performed by heating the polyphenylene ether and the unsaturated carboxylic acid or unsaturated carboxylic anhydride, generally at a temperature in the range of from 100 to 390° C. The reaction can be performed by any of the conventional processes. For example, the reaction can be performed by a solution process in which the reaction is performed in a solution, or a melt-mixing process in which an extruder or the like is used. Of these two types of processes, the melt-mixing process is easier to perform.

The above-mentioned reaction may be performed in the presence of a radical generator. As the radical generator for the reaction, there can be used any of the radical generators mentioned above in connection with the reaction of the precursory modified polyphenylene ether with the phenolic compound.

The amount of the unsaturated carboxylic acid or unsaturated carboxylic anhydride used for the reaction is generally from 0.01 to 20 parts by weight, preferably from 0.01 to 5.0 parts by weight, more preferably from 0.1 to 3.0 parts by weight, relative to 100 parts by weight of the polyphenylene ether.

When the amount of the unsaturated carboxylic acid or unsaturated carboxylic anhydride is less than 0.01 part by weight, the polyphenylene ether is likely to undergo substantially no modification. On the other hand, when the amount of the unsaturated carboxylic acid or unsaturated carboxylic anhydride is more than 20 parts by weight, it is likely that the electric characteristics and heat resistance of the modified polyphenylene ether obtained are impaired.

With respect to each of the acrylic ester and methacrylic ester, it is preferred that the ester contains an alkyl group having 9 to 22 carbon atoms, an alkenyl group having 9 to 22 carbon atoms, an aralkyl group having 9 to 22 carbon atoms, or a cycloalkyl group having 9 to 22 carbon atoms. It is more preferred that the ester contains an alkenyl group having 12 to 18 carbon atoms, an aralkyl group having 12 to 18 carbon atoms, or a cycloalkyl group having 12 to 18 carbon atoms.

With respect to each of the above-mentioned hydrocarbon groups in the acrylic ester and methacrylic ester, when the number of carbon atoms of the hydrocarbon group is 8 or less, disadvantages are likely to occur wherein the appearance and smoothness of a shaped article (such as a curable film) produced using the modified polyphenylene ether are impaired. On the other hand, when the number of carbon atoms of the hydrocarbon group is 23 or more, disadvantages are likely to occur wherein the heat resistance of the shaped article becomes extremely low. When the number of carbon atoms of the hydrocarbon group is in the range of from 9 to 22, the shaped article exhibits not only excellent heat resistance but also excellent smoothness.

The acrylic ester and methacrylic ester are represented by the following formula (9):

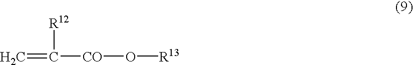

wherein R¹² represents a hydrogen atom or a methyl group, and R¹³ represents an alkyl group, an alkenyl group, an aralkyl group or a cycloalkyl group, each of which is unsubstituted or substituted.

Specific examples of acrylic esters and methacrylic esters include lauryl acrylate, tridecyl acrylate, cetyl acrylate, stearyl acrylate, isobornyl acrylate, phenoxydiethylene glycol acrylate, phenoxypolyethylene glycol acrylate, 2-acryloyloxyethyl-2-hydroxypropylphthalate, 2-hydroxy-3-phenoxypropyl acrylate, lauryl methacrylate, tridecyl methacrylate, stearyl methacrylate, morpholinoethyl methacrylate, methacrylate having, addition-bonded thereto, tribromophenol-3-ethyleneoxide, cyclohexyl methacrylate and methoxypolyethylene glycol methacrylate.

The reaction of the polyphenylene ether with at least one vinyl compound selected from the group consisting of an acrylic ester and a methacrylic ester can be performed by heating the polyphenylene ether and the vinyl compound to a temperature which is equal to or higher than the glass transition temperature of the polyphenylene ether, in the absence or presence of a radical generator.

As the radical generator for the reaction of the polyphenylene ether with the vinyl compound, there can be used any of the radical generators mentioned above in connection with the reaction of the precursory modified polyphenylene ether with the phenolic compound.

The amount of the at least one vinyl compound selected from the group consisting of an acrylic ester and a methacrylic ester is generally from 0.01 to 20 parts by weight, preferably from 0.1 to 20 parts by weight, more preferably from 1.0 to 5.0 parts by weight, relative to 100 parts by weight of the polyphenylene ether. When the amount of the vinyl compound is less than 0.01 part by weight, there is a tendency that the polyphenylene ether undergoes substantially no modification. On the other hand, when the amount of the vinyl compound is more than 20 parts by weight, there is a tendency that the electric characteristics and heat resistance of the modified polyphenylene ether obtained are impaired.

As described above, the modified polyphenylene ether of the present invention is obtained by reacting a precursory modified polyphenylene ether with a phenolic compound in the presence of a radical generator.

In the present invention, the term “phenolic compound” means a compound having 1 or more phenolic hydroxyl groups.

Specific examples of phenolic compounds include 2-methylphenol, 3-methylphenol, 4-methylphenol, 2,3-xylenol, 2,4-xylenol, 2,5-xylenol, 2,6-xylenol, 2,4,6-trimethylphenol, 2-methyl-6-allylphenol, 2-methoxyphenol, 3-methoxyphenol, 4-methoxyphenol, 2-methoxy-4-allylphenol, 2-allylphenol, bisphenols (such as bisphenol A, bisphenol F and bisphenol S), a phenolic novolak and a cresolic novolak. Of these phenolic compounds, preferred are 2,3-xylenol, 2,4-xylenol, 2,5-xylenol, 2,6-xylenol, bisphenols, a phenolic novolak and a cresolic novolak.

The amount of the phenolic compound is preferably from 0.1 to 20 parts by weight, more preferably from 0.5 to 10 parts by weight, relative to 100 parts by weight of the precursory modified polyphenylene ether.

When the amount of the phenolic compound is less than 0.1 part by weight, there is a tendency that the below-mentioned redistribution reaction does not easily advance, so that it becomes difficult to obtain a modified polyphenylene ether having a desired number average molecular weight. On the other hand, when the amount of the phenolic compound is more than 20 parts by weight, there is a tendency that the redistribution reaction advances too vigorously, making it difficult to control the redistribution.

The reason why the modified polyphenylene ether of the present invention exhibits excellent melt properties, excellent mechanical properties and excellent film-forming properties is presumed to be as follows.

The modified polyphenylene ether of the present invention is obtained by reacting a precursory modified polyphenylene ether with a phenolic compound in the presence of a radical generator, wherein the precursory modified polyphenylene ether comprises a plurality of polyphenylene ether chains each having a terminal phenolic hydroxyl group, wherein the plurality of polyphenylene ether chains collectively have at least one terminal phenolic hydroxyl group modified and wherein, however, not all of the terminal phenolic hydroxyl groups of the plurality of polyphenylene ether chains are modified.

Among the above-mentioned polyphenylene ether chains, the polyphenylene ether chains each having a terminal phenolic hydroxyl group which is not modified undergo a redistribution reaction with the phenolic compound, so that such polyphenylene ether chains are cleaved successively from the terminals thereof. As a result, the number average molecular weight of the modified polyphenylene ether is lowered.

On the other hand, the polyphenylene ether chains each having a terminal phenolic hydroxyl group which is modified do not undergo a redistribution reaction with the phenolic compound, so that such polyphenylene ether chains remain uncleaved. As a result, the modified polyphenylene ether retains high molecular weight components.

Therefore, the modified polyphenylene ether, which is obtained by reacting the precursory modified polyphenylene ether with the phenolic compound, has a melt viscosity which has been lowered in accordance with the lowering of the number average molecular weight thereof, so that the modified polyphenylene ether exhibits excellent melt properties. Further, since the modified polyphenylene ether retains high molecular weight components, the modified polyphenylene ether exhibits excellent mechanical properties and excellent film-forming properties.

With respect to the curable polyphenylene ether resin composition of the present invention, an explanation is made below.

The curable polyphenylene ether resin composition of the present invention comprises the modified polyphenylene ether of the present invention and a thermocurable resin. The amounts of the modified polyphenylene ether and the thermocurable resin are respectively 1 to 99 parts by weight and 99 to 1 parts by weight, preferably respectively 10 to 70 parts by weight and 90 to 30 parts by weight, relative to 100 parts by weight of the total of the modified polyphenylene ether and the thermocurable resin. When the amount of the thermocurable resin is less than 1 part by weight, disadvantages are likely to occur wherein, when the curable polyphenylene ether resin composition is softened by heating, the resin composition has unsatisfactory melt properties. Therefore, when such resin composition is used to form an insulating layer on a circuit layer of a circuit board, it becomes impossible to completely fill the interstices in the circuit layer with the resin composition, and also the adhesion between the circuit layer and the insulating layer becomes poor. On the other hand, when the amount of the thermocurable resin is more than 99 parts by weight, the dielectric constant and dielectric loss tangent of the curable polyphenylene ether resin composition become disadvantageously high.

Preferred examples of thermocurable resins used in the curable polyphenylene ether resin composition include triallyl isocyanurate, triallyl cyanurate, cyanate ester resins, epoxy resins, benzoxazine resins, acrylic resins, phenolic resins and diallyl phthalate resins. These resins can be used individually or in combination. Of these resins, especially preferred are triallyl isocyanurate, triallyl cyanurate, cyanate ester resins, epoxy resins and benzoxazine resins.

In the present invention, a curing agent or a cure accelerating agent can be used for the thermocurable resin so long as the properties of the thermocurable resin are not impaired. Preferred examples of such agents for epoxy resins include dicyandiamide, imidazole and phenolic resins. Preferred examples of such agents for triallyl isocyanurate or triallyl cyanurate include radical generators. Examples of radical generators include not only the above-exemplified radical generators, but also dialkylperoxides, such as α,α′-bis (t-butylperoxy)diisopropylbenzene, dicumylperoxide, 2,5-dimethyl-2,5-bis(t-butylperoxy)hexane, t-butylcumylperoxide, di-t-butylperoxide, and 2,5-dimethyl-2,5-bis(t-butylperoxy)-3-hexyne.

It is preferred to use the curing agent or cure accelerating agent in an amount of from 0.01 to 15 parts by weight, relative to 100 parts by weight of the thermocurable resin. When the amount of the agent is less than 0.01 part by weight, it is likely that the agent cannot exhibit its desired effects. On the other hand, when the amount of the agent is more than 15 parts by weight, disadvantages are likely to occur wherein the curing of the thermocurable resin becomes too vigorous and, hence, the resultant cured resin becomes brittle.

In the present invention, an inorganic filler or an organic filler may be incorporated into the curable polyphenylene ether resin composition in order to achieve advantages such that the strength is improved with respect not only to the insulating layer formed from the resin composition but also to the circuit board containing the insulating layer, that the thermal expansion coefficient of the insulating layer is lowered, and that the insulating layer has good thermal conductivity. Examples of inorganic fillers include SiO₂, A₁₂O₃, ZrO₂, TiO₂, AlN and SiC. Examples of organic fillers include aramid fibers and carbon fibers.

Conventional additives may be incorporated into the curable polyphenylene ether resin composition of the present invention so long as the properties of the resin composition are not impaired. Examples of conventional additives include inorganic fillers, such as barium sulfate, calcium carbonate, barium titanate, silicon dioxide powder, amorphous silica, talc, clay and mica powder; auxiliary flame retardants, such as antimony(III) oxide and antimony(V) oxide; organic fillers, such as silicone powder, nylon powder and fluororesin powder; adhesion-improving agents, such as those containing imidazole, thiazole or triazole, and silane coupling agents. If desired, there may be used a conventional dye, such as phthalocyanine blue, phthalocyanine green, iodine green, disazo yellow, titanium oxide or carbon black. For improving the flame retardancy of the curable polyphenylene ether resin composition, there may be used flame retardants or auxiliary flame retardants, such as those containing a chlorine atom, a bromine atom, a phosphorus atom, a nitrogen atom or a silicon atom.

With respect to the curable film and cured film of the present invention, explanations are made below.

The curable film of the present invention comprises the curable polyphenylene ether resin composition of the present invention.

Examples of methods for forming a curable film from the curable polyphenylene ether resin composition of the present invention include a method in which the curable polyphenylene ether resin composition is dissolved in a solvent, such as an aromatic hydrocarbon (e. g., toluene or xylene), and the resultant varnish is formed into a film by a doctor blade method or the like; and a method in which the curable polyphenylene ether resin composition is subjected to an extrusion molding using an extruder having a T-die or to compression molding, thereby obtaining a film. If desired for achieving a predetermined film thickness, a film which has been obtained by molding the resin composition may be further subjected to an additional treatment, such as a calender roll treatment, or to a compression molding.

It is preferred that the molding of the curable polyphenylene ether resin composition is performed at a temperature of from 80 to 300° C. under a pressure of from 0.1 to 1,000 kgf/cm for a period of from 1 minute to 10 hours, more advantageously at a temperature of from 150 to 250° C. under a pressure of from 1 to 500 kgf/cm² for a period of from 1 minute to 5 hours.

The thickness of the curable film is preferably from 1 to 500 μm, more preferably from 10 to 100 μm.

The cured film of the present invention is obtained by curing the curable film of the present invention. With respect to the method for curing the curable film, there is no particular limitation. It is preferred that the curing is performed at a temperature of from 80 to 300° C. under a pressure of from 0.1 to 1,000 kgf/cm² for a period of from 1 minute to 10 hours, more advantageously at a temperature of from 150 to 250° C. under a pressure of from 1 to 500 kgf/cm² for a period of from 1 minute to 5 hours.

With respect to the curable structure and cured structure of the present invention, explanations are made below.

The curable structure of the present invention comprises an insulating layer of the curable film of the present invention and, disposed thereon, an electroconductive layer of a metallic foil.

Examples of metallic foils include a copper foil and an aluminum foil. With respect to the thickness of the metallic foil, there is no particular limitation; however, generally, the thickness of the metallic foil is preferably from 1 to 200 μm, more preferably from 5 to 100 μm.

For improving the adhesion of the metallic foil to the insulating layer, the metallic foil may, prior to the use thereof for producing a curable structure, be subjected first to a mechanical abrasion treatment, such as sanding using a sand paper or a sand cloth, wet blasting or dry blasting, and then to degreasing, etching, Alumite-forming treatment (i.e., anodization of aluminum), chemical coating or the like.

With respect to the method for producing the curable structure of the present invention, there is no particular limitation. As examples of methods for producing the curable structure, there can be mentioned the following three methods. In a first method (method 1), the curable polyphenylene ether resin composition of the present invention is uniformly dissolved in a solvent to obtain a varnish, and the varnish is applied to a metallic foil to form a coating on the metallic foil, thereby obtaining a curable structure. In a second method (method 2), the curable film of the present invention is disposed on a metallic foil, and the resultant is subjected to melt pressing to thereby obtain a curable structure. In a third method (method 3), a metallic foil is formed on the curable film of the present invention by vacuum evaporation, sputtering, electroless plating or the like to thereby obtain a curable structure. Of these three methods, from the viewpoint of ease in operation, method 1 is preferred.

The cured structure of the present invention is obtained by curing the curable structure of the present invention. The conditions for curing the curable structure are the same as those employed for curing the curable film of the present invention to obtain the cured film of the present invention.

A curable laminate can be produced by a method comprising forming a circuit in the electroconductive layer of the curable structure of the present invention to obtain a curable structure having a circuit in the electroconductive layer thereof, and laminating a predetermined number of such curable structures. When it is desired to obtain a curable laminate having an insulating layer which has a thickness larger than that of the insulating layer of the curable structure, such curable laminate can be obtained by interposing a predetermined number of curable films of the present invention between adjacent curable structures in the curable laminate.

A curable laminate can be produced by a method in which a predetermined number of the curable structures are put one upon another, or a predetermined number of the curable structures and a predetermined number of the curable films are put one upon another and/or alternately arranged, and the resultant is subjected to heat pressing or the like at a temperature at which the heat pressing to form a laminate can be effected, but a curing of the curable resin composition does not advance. Alternatively, a curable laminate can also be produced by a method in which a cycle, wherein curable structures are put one upon another, or curable structure (s) and curable film(s) are put one upon another and/or alternately arranged, followed by effecting heat pressing or the like at the above-mentioned temperature, is repeated until a curable laminate having a predetermined number of layers is obtained. The pressing temperature is generally from 50 to 200° C.

A cured laminate can be obtained by curing the curable laminate. The conditions for curing the curable laminate are the same as those employed for curing the curable film of the present invention. A cured laminate can also be obtained by a method in which a cycle comprising laminating two or more layers and effecting curing is repeated until a cured laminate having a predetermined number of layers is obtained.

With respect to the curable composite material of the present invention and the cured composite material of the present invention, explanations are made below.

The curable composite material of the present invention comprises a substrate and the curable polyphenylene ether resin composition of the present invention. Examples of materials for the substrate used in the curable composite material include woven or non-woven glass cloths, such as a roving cloth, a cloth, a chopped strand mat and a surfacing mat; metallic fiber cloths; and synthetic or natural inorganic fiber cloths; woven or non-woven cloths produced from synthetic fibers, such as polyvinyl alcohol fibers, polyester fibers, acrylic fibers, aromatic polyamide fibers and; natural fiber cloths, such as a cotton cloth, a hemp cloth and a felt; carbon fiber cloths; natural cellulose cloths, such as a kraft paper, a cotton paper and a glass-mixed paper. These materials can be used individually or in combination.

The amount of the substrate is preferably from 5 to 90 parts by weight, more preferably from 10 to 80 parts by weight, most preferably from 20 to 70 parts by weight, relative to 100 parts by weight of the curable composite material. When the amount of the substrate is less than 5 parts by weight, the dimensional stability and strength of the cured composite material obtained by curing the curable composite material tend to be unsatisfactory. On the other hand, when the amount of the substrate is more than 90 parts by weight, the dielectric characteristics and flame retardancy of the curable composite material tend to be poor.

If desired, in the curable composite material of the present invention, a coupling agent may be used for the purpose of improving the adhesion at the interface between the polyphenylene ether resin composition and the substrate. With respect to the coupling agent, there is no particular limitation; however, illustrative examples of coupling agents include a silane coupling agent, a titanate-containing coupling agent, an aluminum-containing coupling agent and a zirconium aluminate-containing coupling agent.

With respect to the method for producing the curable composite material of the present invention, there is no particular limitation. Examples of methods for producing the curable composite material include a method in which the resin composition of the present invention and optionally additional material(s) are uniformly dissolved or dispersed in a solvent, such as a halide, an aromatic compound, ketone or a mixture thereof; and the resultant solution or dispersion is impregnated into a substrate, followed by drying.

The impregnation of the substrate with the solution or dispersion can be conducted by dipping, coating or the like. If desired, the impregnation can be conducted a plurality of times. When the impregnation is conducted a plurality of times, a plurality of solutions or dispersions having different compositions and concentrations can be used so as to obtain a curable composite material having a desired resin composition and resin amount.

The cured composite material of the present invention is obtained by curing the curable composite material of the present invention, wherein the curing is conducted by heating or the like. In the present invention, as the curable composite material, either a single curable composite material or a plurality of curable composite materials may be used.

With respect to the method for producing the cured composite material of the present invention, there is no particular limitation. Examples of methods for producing the cured composite material include a method in which a plurality of curable composite materials of the present invention are put one upon another, and the resultant laminate is subjected to heat pressing to effect bonding between the curable composite materials and also effect curing of the curable composite materials, thereby obtaining a cured composite material having a desired thickness. Further, the thus obtained cured composite material may be used in combination with the curable composite material in order to produce another cured composite material.

In general, the pressing and curing of the above-mentioned laminate for producing the cured composite material of the present invention are simultaneously performed by heat pressing or the like.

However, the pressing and curing of the laminate for producing the cured composite material of the present invention may be separately performed. For example, there can be used a method in which a laminate of curable or semi-cured composite materials is first obtained and then the laminate is cured by heat treatment or the like.

It is preferred that the pressing and curing are performed at a temperature of from 80 to 300° C. under a pressure of from 0.1 to 1,000 kgf/cm² for a period of from 1 minute to 10 hours, more advantageously at a temperature of from 150 to 250° C. under a pressure of from 1 to 500 kgf/cm² for a period of from 1 minute to 5 hours.

With respect to a curable composite structure and a cured composite structure, explanations are made below.

In the present invention, the term “curable composite structure” means a structure which is obtained by putting one upon another the curable composite material of the present invention and at least one member selected from the group consisting of the curable film of the present invention and the above-mentioned curable laminate. The term “cured composite structure” means a structure which is obtained by curing the curable composite structure, wherein the curing is conducted by heating or the like.

With respect to the method for producing the curable composite structure and the cured composite structure, there is no particular limitation. As an example of a method for producing the cured composite structure, there can be mentioned a method in which the curable composite material and at least one member selected from the group consisting of the curable film and the curable laminate are put one upon another, and the resultant composite laminate is subjected to heat pressing to effect bonding between the layers of the composite laminate and also effect curing of the composite laminate, thereby obtaining a cured composite structure. By this method, a cured composite structure having a desired thickness can be obtained.

The curable polyphenylene ether resin composition of the present invention can be advantageously used in the above-mentioned build-up method. With respect to the build-up method performed using the curable polyphenylene ether resin composition of the present invention, explanations are made below.

With respect to the substrate for use in the build-up method, there is no particular limitation. Examples of substrates include a double-sided copper-clad substrate, a single-sided copper-clad substrate, an aluminum substrate and an iron substrate. When a double-sided copper-clad substrate or a single-sided copper-clad substrate is used, a circuit pattern may be previously formed thereon.

Using the substrate, a multi-layered circuit board is obtained by the following method. On the substrate is disposed the curable film of the present invention (which comprises the curable polyphenylene ether resin composition of the present invention) and/or the curable structure of the present invention (which comprises an insulating layer of the curable film and, disposed thereon, an electroconductive layer of a metallic foil), and the resultant is subjected to heat pressing and then, if desired, a circuit pattern may be formed thereon. Then, an additional curable film and/or an additional curable structure is disposed onto the above-mentioned curable film and/or the curable structure, and the resultant is subjected to heat pressing. By repeating a cycle comprising these operations one or more times, a multi-layered circuit board is obtained. In this method, with respect to the curing of each of the curable films by heating, the curing may be conducted simultaneously with the heat pressing. Alternatively, the curing may be conducted after the heat pressing. When the curing of each of the curable films is conducted simultaneously with the heat pressing, it is not necessary to conduct a complete curing. In the case where the curing is incomplete, a complete curing by heating can be conducted after completion of the lamination of all necessary layers of the multi-layered circuit board.

With respect to the method for effectively and efficiently producing the modified polyphenylene ether of the present invention, an explanation is made below.

It is preferred that the production of the modified polyphenylene ether of the present invention having a number average molecular weight of not smaller than 4,000 is conducted by a method which comprises performing a homogeneous reaction of a precursory modified polyphenylene ether with a phenolic compound in the presence of a radical generator, wherein the precursory modified polyphenylene ether comprises a plurality of polyphenylene ether chains each having a terminal phenolic hydroxyl group, wherein the plurality of polyphenylene ether chains collectively have at least one terminal phenolic hydroxyl group modified and wherein, however, not all of the terminal phenolic hydroxyl groups of the plurality of polyphenylene ether chains are modified.

A preferred mode of the above-mentioned method is explained below.

First, the precursory modified polyphenylene ether is completely dissolved in an organic solvent to obtain a solution. When the dissolution of the precursory modified polyphenylene ether in the organic solvent is not complete, it tends to occur that a desired modified polyphenylene ether cannot be obtained. From the viewpoint of acceleration of the dissolution, it is preferred that the dissolution of the precursory modified polyphenylene ether in the organic solvent is conducted at a temperature of from 60 to 120° C.

Next, to the obtained solution is added the phenolic compound to obtain a solution. After it is confirmed that the phenolic compound added has been completely dissolved, a radical generator is added to the solution to obtain a mixture, and a homogeneous reaction of the precursory modified polyphenylene ether with the phenolic compound in the presence of the radical generator is performed, while stirring, at a temperature of from 60 to 120° C. under atmospheric pressure for about 10 to 300 minutes, thereby obtaining a reaction mixture containing a modified polyphenylene ether.

The types and amounts of the precursory modified polyphenylene ether, phenolic compound and radical generator which are used in the homogeneous reaction are the same as those described above in connection with the production of the modified polyphenylene ether of the present invention.

With respect to the organic solvent used in the homogeneous reaction, there is no particular limitation; however, it is preferred to use an aromatic hydrocarbon solvent, such as toluene, benzene or xylene.

By using the reaction mixture containing a modified polyphenylene ether wherein the reaction mixture is obtained by the homogeneous reaction, a varnish of a curable polyphenylene ether resin composition can be produced. For example, a varnish of a curable polyphenylene ether resin composition can be obtained by isolating the modified polyphenylene ether from the reaction mixture, and adding the isolated, modified polyphenylene ether and a thermocurable resin to an organic solvent, followed by mixing, to thereby obtain a curable polyphenylene ether resin composition in a varnish form.

In this method, the method for isolating the modified polyphenylene ether from the reaction mixture is not particularly limited. As a specific example of a method for the isolation, there can be mentioned a method in which a liquid substance which is a poor solvent for the modified polyphenylene ether and which is miscible with the organic solvent is added to the reaction mixture to thereby deposit the modified polyphenylene ether in a powdery (particulate) form, followed by recovering the powder of the modified polyphenylene ether. Examples of liquid substances used for isolating the modified polyphenylene ether from the reaction mixture include water, methanol, ethanol, acetone and a mixture thereof.

Preferred examples of organic solvents to which the isolated, modified polyphenylene ether and a thermocurable resin are added include aromatic hydrocarbons, such as toluene and xylene.

It is preferred that the above-mentioned curable polyphenylene ether resin composition obtained in a varnish form comprises 1 to 99 parts by weight of the modified polyphenylene ether and 99 to 1 part by weight of the thermocurable resin, wherein the total amount of the modified polyphenylene ether and the thermocurable resin is 100 parts by weight.

As the thermocurable resin, there can be used any of the thermocurable resins mentioned above in connection with the curable polyphenylene ether resin composition of the present invention. However, it is preferred that the thermocurable resin is at least one member selected from the group consisting of triallyl isocyanurate, triallyl cyanurate, a cyanate ester resin, an epoxy resin and a benzoxazine resin.

Using the varnish obtained by the above-mentioned method, a curable film can be produced by a method comprising applying the varnish onto a support to obtain a coating, and peeling off the coating from the support to obtain a curable film.

Examples of supports used for producing the curable film include a polyethylene terephthalate (PET) film and a polyethylene naphthalate film.

As an example of a method for applying the varnish onto the support to obtain a coating, there can be mentioned a method using a blade coater. The solidification of the varnish coating formed on the support is generally conducted by drying the varnish coating. It is preferred that the drying temperature is from 20 to 200° C. It is preferred that the drying time is from 1 second to 5 hours.

When the homogeneous reaction of the precursory modified polyphenylene ether with the phenolic compound is performed in the presence of not only a radical generator but also an organic solvent, the reaction mixture (containing the modified polyphenylene ether) obtained by the homogeneous reaction can be used to produce a varnish of a curable polyphenylene ether resin composition. For example, by adding a thermocurable resin to the reaction mixture, a curable polyphenylene ether resin composition can be obtained in a varnish form.

It is preferred that the above-mentioned curable polyphenylene ether resin composition obtained in a varnish form comprises 1 to 99 parts by weight of the modified polyphenylene ether and 99 to 1 part by weight of the thermocurable resin, wherein the total amount of the modified polyphenylene ether and the thermocurable resin is 100 parts by weight.

As the thermocurable resin, there can be used any of the thermocurable resins mentioned above in connection with the curable polyphenylene ether resin composition of the present invention. However, it is preferred that the thermocurable resin is at least one member selected from the group consisting of triallyl isocyanurate, triallyl cyanurate, a cyanate ester resin, an epoxy resin and a benzoxazine resin.

Using the varnish obtained by the above-mentioned method, a curable film can be produced by a method comprising applying the varnish onto a support to obtain a coating, and peeling off the coating from the support to obtain a curable film.

Examples of supports used for producing the curable film include a polyethylene terephthalate (PET) film and a polyethylene naphthalate film.

As an example of a method for applying the varnish onto the support to obtain a coating, there can be mentioned a method using a blade coater. The solidification of the varnish coating formed on the support is generally conducted by drying the varnish coating. It is preferred that the drying temperature is from 20 to 200° C. It is preferred that the drying time is from 1 second to 5 hours.

### BEST MODE FOR CARRYING OUT THE INVENTION

Hereinbelow, the present invention will be described in more detail with reference to Synthesis Examples, Examples and Comparative Examples, but they should not be construed as limiting the scope of the present invention.

In the Synthesis Examples, Examples and Comparative Examples, various properties were measured and evaluated by the following methods.

(1) Number average molecular weight, weight average molecular weight and Z-average molecular weight of a polymer:

A sample solution of a polymer is prepared by dissolving 50 mg of the polymer in 50 ml of chloroform. The sample solution is subjected to gel permeation chromatography (GPC), and the number average molecular weight of the polymer is determined using a calibration curve obtained with respect to standard polystyrene samples. The GPC is conducted under the following conditions:

GPC apparatus: HLC-8020GPC (manufactured and sold by TOSOH Corporation, Japan);

Columns: TSKGEL G2500HXL, TSKGEL G3000HXL, TSKGEL G4000HXL and TSKGEL G5000HXL (each of which is manufactured and sold by TOSOH Corporation, Japan), connected in series;

Mobile phase: chloroform (flow rate: 1 ml/min);

Detector: 8010 type UV detector (manufactured and sold by TOSOH Corporation, Japan) (wave length: 254 nm); and

Data processor: SC-8020 (manufactured and sold by TOSOH Corporation, Japan).

The number average molecular weight (Mn), weight average molecular weight (Mw) and Z-average molecular weight (Mz) of the polymer are, respectively, defined by the below-mentioned formulae (for detailed information, reference can be made to “Encyclopedia of Polymer Science and Engineering”, Volume 10, page 1, published by John Wiley & Sons, U.S.A., 1985).

Mn=(ΣMᵢNᵢ)/(ΣNᵢ)

Mw=(ΣMᵢ²Nᵢ)/(ΣMᵢNᵢ)

Mz=(ΣMᵢ³Nᵢ)/(ΣMᵢNᵢ)

wherein Nᵢ represents the number of molecules each having a molecular weight of Mᵢ.

(2) Methylene chloride resistance of a cured film

From a cured structure comprising a cured film and, disposed thereon, a copper foil, the copper foil is removed. From the cured film is cut out a sample having a size of 60 mm×60 mm. The sample is boiled in 500 ml of methylene chloride for 5 minutes. Then, the sample is visually observed and the appearance of the sample is evaluated by the following criteria.

◯: the sample has a good appearance.

x: the sample has suffered chalking and the interior thereof is exposed.

(3) Dielectric constant and dielectric loss tangent of a cured film

Using an impedance analyzer HP-4192 (manufactured and sold by Yokokawa-Hewlett-Packard, Ltd., Japan), the dielectric constant and dielectric loss tangent are measured at 1 MHz.

(4) Soldering heat resistance of a cured film

From a cured structure comprising a cured film and, disposed thereon, a copper foil, a sample having a size of 60 mm×60 mm is cut out. From the sample is removed a half of the copper foil. Then, the sample (copper foil-partially removed cured structure) is floated on a solder bath having a temperature of 260° C. for 120 seconds. Then, the sample is visually observed and the appearance of the sample is evaluated by the following criteria.

◯: the appearance of the sample has not changed.

x: the copper foil of the sample has suffered blister and delamination.

(5) Glass transition temperature and thermal expansion coefficient of a cured film

From a cured structure comprising a cured film and, disposed thereon, a copper foil, the copper foil is removed. From the cured film is cut out a rectangular sample having a size of 3 mm×3 cm. Using TMA-10 apparatus (manufactured and sold by Seiko Instrument Inc., Japan), the glass transition temperature and thermal expansion coefficient of the sample are determined by the TMA method (thermal mechanical analysis), wherein the measuring mode is a tensile mode in which the distance between the chucks is 1 cm.

(6) Melt viscosity of a resin composition

Films of a resin composition are laminated so as to obtain a total thickness of 0.5 mm. The resultant laminate is used as a sample as follows. The sample is sandwiched between a pair of parallel plates each having a diameter of 25 mm. A portion of the sample, which is not sandwiched by the plates, is cut off along the periphery of each plate by means of a razor so that the sample has the same shape as that of each plate (circular shape of a 25 mm diameter). Then, the melt viscosity of the sample is measured by means of RMS-800 apparatus (manufactured and sold by Rheometric Scientific, Inc., U.S.A.). The measurement is conducted under conditions wherein the measuring frequency is 10 Hz, and the temperature is elevated from 100 to 200° C. at a rate of 7° C./min. The obtained lowest value of the viscosity is defined as the melt viscosity of the resin composition.

(7) Interstice-filling properties of a curable film There is provided a circuit board comprising a substrate having formed thereon a circuit comprised of a copper foil having a thickness of 35 μm. A curable film (comprising a curable resin composition) having a thickness of 70 μm is laminated on the circuit of the circuit board. The resultant, film-laminated circuit board is subjected to a heat pressing at 200° C. under a pressure of 5 kgf/cm₂ for 1 hour using a vacuum press machine (Hot Press VH2-1239, manufactured and sold by KITAGAWA SEIKI Co., Ltd., Japan) to cure the curable film on the circuit of the circuit board, thereby obtaining a circuit board with the circuit having thereon an insulating layer comprised of the resultant cured film. Then, the circuit board with the copper foil circuit having thereon the insulating layer thereon is embedded in an epoxy resin, and the circuit board embedded in the epoxy resin is cut together with the resin in a manner wherein the copper foil circuit having thereon the insulating layer is thicknesswise cut, thereby exposing the vertical cross-section of the copper foil circuit of the circuit board. The cross-section is abraded. The abraded cross-section is observed through a microscope (PME3, manufactured and sold by Olympus Optical Co., Ltd., Japan). When it is observed that the interstices of the circuit comprised of the copper foil are completely filled with the insulating layer comprised of the cured film, the curable film employed is defined as having good interstice-filling properties. On the other hand, when the interstices of the circuit comprised of the copper foil are not completely filled with the insulating layer comprised of the cured film, the curable film employed is defined as having poor interstice-filling properties.

Four different types of precursory modified polyphenylene ether were synthesized as follows.

### Synthesis Example 1

100 Parts by weight of a poly(2,6-dimethyl-1,4-phenylene ether) having a number average molecular weight of 18,000, 1.5 parts by weight of maleic anhydride and 1.0 part by weight of 2,5-dimethyl-2,5-di(t-butylperoxy) hexane (Perhexa 25B, manufactured and sold by Nippon Oil & Fats Co., Ltd., Japan) were dry-blended at room temperature. The resultant mixture was then extruded using a twin-screw extruder (Twin Screw Extruder ZSK-70SC, manufactured and sold by Krupp Werner & Pfleiderer GmbH, Japan) under conditions wherein the cylinder temperature was 300° C., the screw temperature was 300° C. and the screw revolution rate was 230 rpm, thereby obtaining a precursory, maleic anhydride-modified polyphenylene ether. Hereinafter, this precursory modified polyphenylene ether is referred to as “reaction product A”.

With respect to reaction product A, the number average molecular weight, the weight average molecular weight and the Z-average molecular weight were measured by the above-mentioned method. The results are shown in Table 1.

### Synthesis Example 2

100 Parts by weight of a poly(2,6-dimethyl-1,4-phenylene ether) having a number average molecular weight of 20,000 and 1.5 parts by weight of maleic anhydride were dry-blended at room temperature. The resultant mixture was then extruded using a twin-screw extruder (Twin Screw Extruder ZSK-70SC, manufactured and sold by Krupp Werner & Pfleiderer GmbH, Japan) under conditions wherein the cylinder temperature was 300° C., the screw temperature was 300° C. and the screw revolution rate was 230 rpm, thereby obtaining a precursory, maleic anhydride-modified polyphenylene ether. Hereinafter, this precursory modified polyphenylene ether is referred to as “reaction product B”.

With respect to reaction product B, the number average molecular weight, the weight average molecular weight and the Z-average molecular weight were measured by the above-mentioned method. The results are shown in Table 1.

### Synthesis Example 3

100 Parts by weight of a poly(2,6-dimethyl-1,4-phenylene ether) having a number average molecular weight of 14,000 and 2.5 parts by weight of stearyl acrylate were blended. The resultant mixture was then extruded using a twin-screw extruder (Twin Screw Extruder ZSK-70SC, manufactured and sold by Krupp Werner & Pfleiderer GmbH, Japan) under conditions wherein the cylinder temperature was 300° C., the screw temperature was 300° C. and the screw revolution rate was 250 rpm, thereby obtaining a precursory, stearyl acrylate-modified polyphenylene ether. Hereinafter, this precursory modified polyphenylene ether is referred to as “reaction product F”.

With respect to reaction product F, the number average molecular weight, the weight average molecular weight and the Z-average molecular weight were measured by the above-mentioned method. The results are shown in Table 3.

### Synthesis Example 4

100 Parts by weight of a poly(2,6-dimethyl-1,4-phenylene ether) having a number average molecular weight of 14,000 and 2.5 parts by weight of stearyl methacrylate were blended. The resultant mixture was then extruded using a twin-screw extruder (Twin Screw Extruder ZSK-70SC, manufactured and sold by Krupp Werner & Pfleiderer GmbH, Japan) under conditions wherein the cylinder temperature was 300° C., the screw temperature was 300° C. and the screw revolution rate was 250 rpm, thereby obtaining a precursory, stearyl methacrylate-modified polyphenylene ether. Hereinafter, this precursory modified polyphenylene ether is referred to as “reaction product G”.

With respect to reaction product G, the number average molecular weight, the weight average molecular weight and the Z-average molecular weight were measured by the above-mentioned method. The results are shown in Table 3.

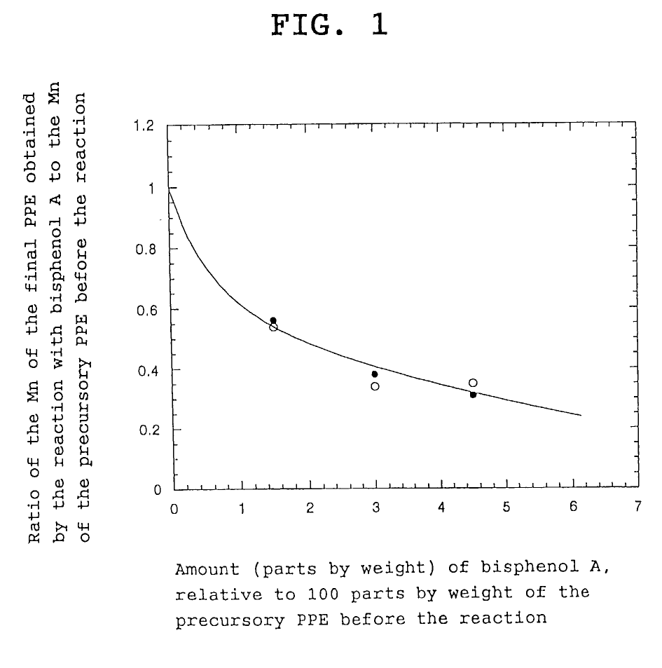

### Explanations on FIGS. 1 to 3

FIGS. 1, 2 and 3 respectively show curves (plotted by indications “●”) obtained by plotting the ratios of changes of the number average molecular weight (Mn), weight average molecular weight (Mw) and Z-average molecular weight (Mz) of a modified polyphenylene ether (PPE) against the amount of the phenolic compound (bisphenol A), wherein the curves are based on the results of Examples 1 to 3. Further, FIGS. 1, 2 and 3 also respectively show curves (plotted by indications “◯”) obtained by plotting the ratios of changes of the number average molecular weight (Mn), weight average molecular weight (Mw) and Z-average molecular weight (Mz) of an unmodified polyphenylene ether (PPE) against the amount of the phenolic compound (bisphenol A), wherein the curves are based on the results of Comparative Examples 1 to 3, provided that FIG. 1 shows a single curve, showing that the ratio of change of the number average molecular weight of the modified PPE in Examples 1 to 3 was substantially the same as the ratio of change of the number average molecular weight of the unmodified PPE in Comparative Examples 1 to 3.

With respect to each of FIGS. 1 to 3, a more specific explanation is made below. FIG. 1 shows a curve obtained by plotting the ratio of change of the number average molecular average weight (Mn) of the polyphenylene ether against the amount of bisphenol A, wherein the curve is based on the results of Examples 1 to 3 and Comparative Examples 1 to 3. In FIG. 1, the abscissa shows the amount (parts by weight) of bisphenol A, relative to 100 parts by weight of the polyphenylene ether before the reaction (precursory PPE), and the ordinate shows the ratio of the number average molecular weight (Mn) of the final polyphenylene ether obtained by the reaction with bisphenol A to the number average molecular weight (Mn) of the polyphenylene ether before the reaction (precursory PPE). Each ● represents the data obtained in Example 1, 2 or 3, and each ◯ represents the data obtained in Comparative Example 1, 2 or 3.

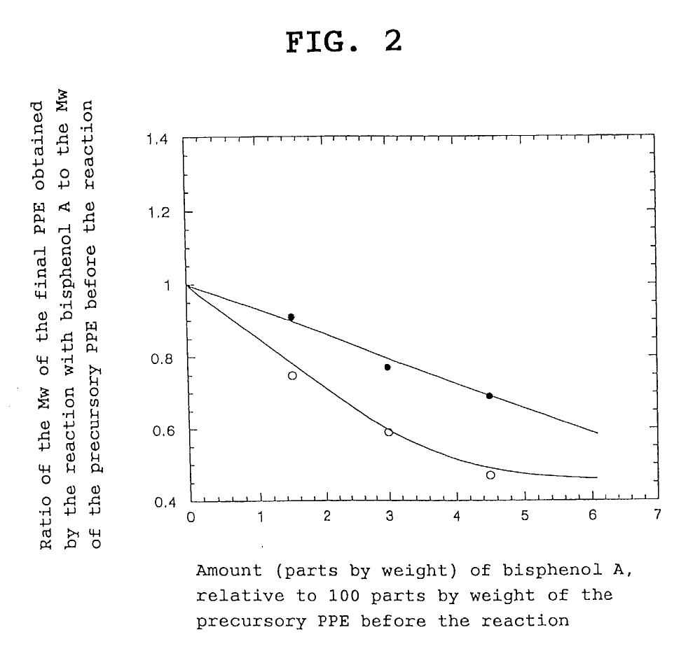

FIG. 2 shows two curves obtained by plotting the ratio of change of the weight average molecular weight (Mw) of the polyphenylene ether against the amount of bisphenol A, wherein the two curves are, respectively, based on the results of Examples 1 to 3 and Comparative Examples 1 to 3. In FIG. 2, the abscissa shows the amount (parts by weight) of bisphenol A, relative to 100 parts by weight of the polyphenylene ether before the reaction, and the ordinate shows the ratio of the weight average molecular weight (Mw) of the final polyphenylene ether obtained by the reaction with bisphenol A to the weight average molecular weight (Mw) of the polyphenylene ether before the reaction. Each ● represents the data obtained in Example 1, 2 or 3, and each ◯ represents the data obtained in Comparative Example 1, 2 or 3.

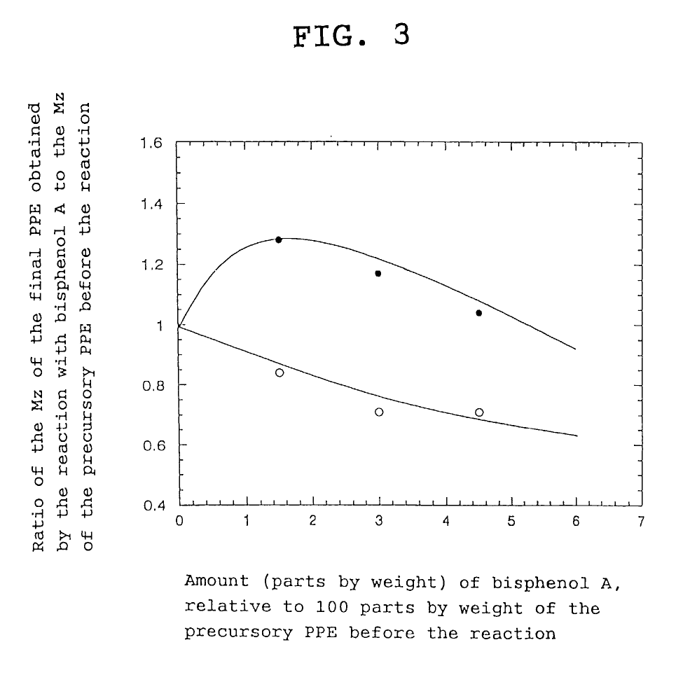

FIG. 3 shows two curves obtained by plotting the ratio of change of the Z-average molecular weight (Mz) of the polyphenylene ether against the amount of bisphenol A, wherein the two curves are, respectively, based on the results of Examples 1 to 3 and Comparative Examples 1 to 3. In FIG. 3, the abscissa shows the amount (parts by weight) of bisphenol A, relative to 100 parts by weight of the polyphenylene ether before the reaction, and the ordinate shows the ratio of the Z-average molecular weight (Mz) of the final polyphenylene ether obtained by the reaction with bisphenol A to the Z-average molecular weight (Mz) of the polyphenylene ether before the reaction. Each ● represents the data obtained in Example 1, 2 or 3, and each ◯ represents data obtained in Comparative Example 1, 2 or 3.

### EXAMPLES 1 to 5

### Production of Maleic Anhydride-modified Polyphenylene Ethers Having Molecular Weights Lowered by Redistribution

In each of Examples 1 to 5, a precursory modified polyphenylene ether, a phenolic compound, a radical generator (bis(4-t-butylcyclohexyl)peroxy dicarbonate; PEROYL TCP, manufactured and sold by Nippon Oil & Fats Co., Ltd., Japan) and an organic solvent (toluene), each of which is described in Table 1, were mixed together in ratios indicated in Table 1 (the values in Table 1 are weight ratios), and a homogeneous reaction of the precursory modified polyphenylene ether with the phenolic compound was performed at 80° C. under atmospheric pressure for 3 hours while stirring.

The resultant reaction product was precipitated using methanol in the same volume as that of the toluene used. The precipitated product was washed with methanol in a volume three times the volume of the methanol used for the precipitation, followed by drying, thereby obtaining a maleic anhydride-modified polyphenylene ether. With respect to the modified polyphenylene ether obtained, the number average molecular weight, the weight average molecular weight and the Z-average molecular weight were measured by the above-mentioned method. The results are shown in Table 1.

From FIGS. 1 to 3 which show the results of Examples 1 to 3, it is apparent that the larger the amount of the bisphenol A, the smaller the number average molecular weight (Mn) of the final, modified polyphenylene ether.

This is also true with respect to the weight average molecular weight (Mw) of the modified polyphenylene ether. However, the decrease of the weight average molecular weight (Mw) of the modified polyphenylene ether is small, as compared to that of the number average molecular weight (Mn) of the modified polyphenylene ether. Further, with respect to the Z-average molecular weight (Mz) of the modified polyphenylene ether, the ratio of change of the Z-average molecular weight (Mz) is small even when the amount of the bisphenol A is increased. These results concerning the weight average molecular weight and Z-average molecular weight of the modified polyphenylene ether show that the final, modified polyphenylene ether retains a large amount of high molecular weight components.

### Comparative Examples 1 to 3

In each of Comparative Examples 1 to 3, an unmodified polyphenylene ether, a phenolic compound, a radical generator (bis(4-t-butylcyclohexyl)peroxy dicarbonate; PEROYL TCP, manufactured and sold by Nippon Oil & Fats Co., Ltd., Japan) and an organic solvent (toluene), each of which is described in Table 1, were mixed together in ratios indicated in Table 1 (the values in Table 1 are weight ratios), and a homogeneous reaction of the unmodified polyphenylene ether with the phenolic compound was performed at 80° C. under atmospheric pressure for 3 hours while stirring.

The resultant reaction product was precipitated using methanol in the same volume as that of the toluene used. The precipitated product was washed with methanol in a volume three times the volume of the methanol used for the precipitation, followed by drying, thereby obtaining an unmodified polyphenylene ether having a number average molecular weight lower than that of the unmodified polyphenylene ether used as a raw material. With respect to the unmodified polyphenylene ether obtained, the number average molecular weight, the weight average molecular weight and the Z-average molecular weight were measured by the above-mentioned method. The results are shown in Table 1.

From FIGS. 1 to 3 which show the results of Comparative Examples 1 to 3, it is apparent that all of the number average molecular weight, weight average molecular weight and Z-average molecular weight of the final, unmodified polyphenylene ether are greatly lowered in accordance with the increase of the amount of the phenolic compound (bisphenol A). These results show that the final, unmodified polyphenylene ether does not contain a large amount of high molecular weight components.

### EXAMPLES 6 to 13

In each of Examples 6 to 10, 12 and 13, a composition having a formulation indicated Table 2 (the values in Table 2 are weight ratios) was added to an organic solvent (toluene) at a temperature of 80° C., and the resultant mixture was stirred for 1 hour to thereby obtain a varnish of a curable polyphenylene ether resin composition. (As shown in Table 2, this curable polyphenylene ether resin composition contains the modified polyphenylene ether produced in Example 1.)

In Example 11, thermocurable resins and cure accelerating agents, each of which is described in Table 2, were added to the reaction mixture (containing a modified polyphenylene ether) obtained by the homogeneous reaction in Example 1, wherein the amounts of the thermocurable resins, cure accelerating agents and modified polyphenylene ether were as indicated in Table 2 (the values in Table 2 are weight ratios), thereby obtaining a varnish of a curable polyphenylene ether resin composition.

In each of Examples 6 to 13, the obtained varnish was applied onto a film of a polyethylene terephthalate (hereinafter referred to as “PET”) having a thickness of 100 μm or onto a matte surface of a copper foil (F2-WS, manufactured and sold by FURUKAWA ELECTRIC CO., LTD., Japan; thickness: 12 μm) by using a blade coater, followed by drying in an air oven at 50° C. for 30 minutes, thereby obtaining a curable film having a thickness of about 70 μm. The obtained curable film was able to be easily completely peeled off from the PET film.

In each of Examples 6 to 13, with respect to the curable film having a thickness of 70 μm, the interstice-filling properties thereof were evaluated by the above-mentioned method. All of the curable films obtained in Examples 6 to 13 exhibited good interstice-filling properties.

In each of Examples 6 to 13, two of the obtained curable films were put one upon another to obtain a film laminate. The film laminate was sandwiched between two copper foils so that the film laminate was contacted with the matte surfaces of the copper foils, to obtain a [copper foil/curable films/copper foil] sandwich. The [copper foil/films/copper foil] sandwich was subjected to heat pressing, thereby preparing a curable laminate comprising two curable films and two copper foils. In the preparation of the curable laminate, the curable films exhibited excellent handling properties and were able to be laminated without suffering breakage.

In each of Examples 6 to 13, the thus obtained curable laminate was cured to thereby obtain a cured laminate. Each of the above-mentioned heat pressing and the curing of the curable laminate was conducted for 1 hour using a vacuum press machine (Hot Press VH2-1239, manufactured and sold by KITAGAWA SEIKI Co., Ltd., Japan) under conditions wherein the pressure was 5 kgf/cm² and the temperature was 200°0 C.

Various properties of the thus obtained cured laminate were measured by the above-mentioned methods.

### Comparative Example 4

A curable film, a curable laminate and a cured laminate were produced in substantially the same manner as in Example 6, except that reaction product A produced in Synthesis Example 1 was used instead of the modified polyphenylene ether produced in Example 1. The obtained curable film was able to be easily completely peeled off from the PET film.

The cured laminate exhibited various good properties. However, the melt viscosity of the polyphenylene ether resin composition was 20,000 poises, which is very high, as compared to that of the polyphenylene ether resin composition obtained in Example 6. With respect to the interstice-filling properties of the curable film, it was observed that voids were present in the interstices of the copper foil circuit, that is, the interstices were not completely filled with the resin composition.

### Comparative Example 5

A curable film was produced in substantially the same manner as in Example 6, except that the unmodified polyphenylene ether produced in Comparative Example 1 was used instead of the modified polyphenylene ether produced in Example 1.

The obtained curable film was fragile and difficult to peel off from the PET film.

### EXAMPLES 14 to 18

### Production of Stearyl Acrylate-modified Polyphenylene Ethers Having Molecular Weights Lowered by Redistribution

In each of Examples 14 to 18, a precursory modified polyphenylene ether, a phenolic compound, a radical generator (bis(4-t-butylcyclohexyl)peroxy dicarbonate; PEROYL TCP, manufactured and sold by Nippon Oil & Fats Co., Ltd., Japan) and an organic solvent (toluene), each of which is described in Table 3, were mixed together in ratios indicated in Table 3 (the values in Table 3 are weight ratios), and a homogeneous reaction of the precursory modified polyphenylene ether with the phenolic compound was performed at 80° C. under atmospheric pressure for 3 hours while stirring.

The resultant reaction product was precipitated using methanol in the same volume as that of the toluene used. The precipitated product was washed with methanol in a volume three times the volume of the methanol used for the precipitation, followed by drying, thereby obtaining a stearyl acrylate-modified polyphenylene ether. With respect to the modified polyphenylene ether obtained, the number average molecular weight, the weight average molecular weight and the Z-average molecular weight were measured by the above-mentioned method. The results are shown in Table 3.

### EXAMPLES 19 to 26

In each of Examples 19 to 23, 25 and 26, a composition having a formulation indicated in Table 4 (the values in Table 4 are weight ratios) was added to an organic solvent (toluene) at a temperature of 80° C., and the resultant mixture was stirred for 1 hour to thereby obtain a varnish of a curable polyphenylene ether resin composition. (As shown in Table 4, this curable polyphenylene ether resin composition contains the modified polyphenylene ether produced in Example 14.)

In Example 24, thermocurable resins and cure accelerating agents, each of which is described in Table 4, were added to the reaction mixture (containing a modified polyphenylene ether) obtained by the homogeneous reaction in Example 14, wherein the amounts of the thermocurable resins, cure accelerating agents and modified polyphenylene ether were as indicated in Table 4 (the values in Table 4 are weight ratios), thereby obtaining a varnish of a curable polyphenylene ether resin composition.

In each of Examples 19 to 26, the obtained varnish was applied onto a PET film having a thickness of 100 μm or onto a matte surface of a copper foil (F2-WS, manufactured and sold by FURUKAWA ELECTRIC CO., LTD., Japan; thickness: 12 μm) using a blade coater, followed by drying in an air oven at 50° C. for 30 minutes, thereby obtaining a curable film having a thickness of about 70 μm. The obtained curable film was able to be easily completely peeled off from the PET film.

In each of Examples 19 to 26, with respect to the curable film having a thickness of 70 μm, the interstice-filling properties thereof were evaluated by the above-mentioned method. All of the curable films obtained in Examples 19 to 26 exhibited good interstice-filling properties.

In each of Examples 19 to 26, two of the obtained curable films were put one upon another to obtain a film laminate. The film laminate was sandwiched between two copper foils so that the film laminate was contacted with the matte surfaces of the copper foils, to obtain a [copper foil/curable films/copper foil] sandwich. The [copper foil/films/copper foil] sandwich was subjected to heat pressing, thereby preparing a curable laminate comprising two curable films and two copper foils. In the preparation of the curable laminate, the curable films exhibited excellent handling properties and were able to be laminated without suffering breakage.

In each of Examples 19 to 26, the thus obtained curable laminate was cured to thereby obtain a cured laminate. Each of the above-mentioned heat pressing and the curing of the curable laminate was conducted for 1 hour using a vacuum press machine (Hot Press VH2-1239, manufactured and sold by KITAGAWA SEIKI Co., Ltd., Japan) under conditions wherein the pressure was 5 kgf/cm² and the temperature was 200° C.

Various properties of the thus obtained cured laminate were measured by the above-mentioned methods.

### Comparative Example 6

A curable film, a curable laminate and a cured laminate were produced in substantially the same manner as in Example 19, except that reaction product F produced in Synthesis Example 3 was used instead of the modified polyphenylene ether produced in Example 14. The obtained curable film was able to be easily completely peeled off from the PET film.

The cured laminate exhibited various good properties. However, the melt viscosity of the polyphenylene ether resin composition was 20,000 poises, which is very high, as compared to that of the polyphenylene ether resin composition obtained in Example 19. Further, the interstice-filling properties of the curable film were poor.

TABLE 1

| | | | | | | Compara. | Compara. | Compara. |
|---|---|---|---|---|---|---|---|---|
| | Ex. 1 | Ex. 2 | Ex. 3 | Ex. 4 | Ex. 5 | Ex. 1 | Ex. 2 | Ex. 3 |
| PPE | 100 (A) | 100 (A) | 100 (A) | 100 (A) | 100 (B) | 100 (C) | 100 (C) | 100 (C) |
| Phenolic | 1.5 | 3.0 | 4.5 | 0.8 | 1.5 | 1.5 | 3.0 | 4.5 |
| compound | (BPA) | (BPA) | (BPA) | (XY) | (BPA) | (BPA) | (BPA) | (BPA) |
| Radical | 2.5 | 5 | 7.5 | 3 | 2.5 | 2.5 | 5 | 7.5 |
| generator | | | | | | | | |
| Toluene | 550 | 550 | 550 | 550 | 550 | 550 | 550 | 550 |
| Mn | 9600 | 6400 | 5300 | 12000 | 10000 | 9800 | 6200 | 6300 |
| (ratio, relative to | (0.56) | (0.38) | (0.31) | (0.71) | (0.50) | (0.54) | (0.34) | (0.35) |
| precursory PPE) | | | | | | | | |
| Mw | 32000 | 27000 | 24000 | 36000 | 36000 | 24000 | 19000 | 15000 |
| (ratio, relative to | (0.91) | (0.77) | (0.69) | (1.03) | (0.90) | (0.75) | (0.59) | (0.47) |
| precursory PPE) | | | | | | | | |
| Mz | 68000 | 62000 | 55000 | 79000 | 70000 | 43000 | 36000 | 36000 |
| (ratio, relative to | (1.28) | (1.17) | (1.04) | (1.49) | (1.17) | (0.84) | (0.71) | (0.71) |
| precursory PPE) | | | | | | | | |

Notes:
Reaction product A: Precursory modified polyphenylene ether produced in Synthesis Example 1. (Mn: 17000, Mw: 35000, Mz: 53000)
Reaction product B: Precursory modified polyphenylene ether produced in Synthesis Example 2. (Mn: 20000, Mw: 40000, Mz: 60000)
Unmodified PPE C: PPE used in Comparative Examples 1 to 3. (Mn: 18000, Mw: 32000, Mz: 51000)
BPA: Bisphenol A
XY: 2,6-xylenol
Radical generator: PEROYL TCP (manufactured and sold by Nippon Oil & Fats Co., Ltd., Japan)
Ratio, relative to precursory PPE: (molecular weight of final PPE)/(molecular weight of PPE before the reaction)

TABLE 2

| | | | | | | | | | Compara. | Compara. |
|---|---|---|---|---|---|---|---|---|---|---|
| | Ex. 6 | Ex. 7 | Ex. 8 | Ex. 9 | Ex. 10 | Ex. 11 | Ex. 12 | Ex. 13 | Ex. 4 | Ex. 5 |
| PPE | 60 (D) | 70 (D) | 50 (D) | 60 (D) | 30 (D) | 40 (D) | 30 (D) | 45 (D) | 60 (A) | 60 (E) |
| Thermocurable | 40 | 30 | 50 | 40 | 70 | 30 | 70 | 55 | 40 | 40 |
| resin | (TAIC) | (TAIC) | (TAIC) | (TAIC) | (AER260) | (AER260) | (B-10) | (B-a) | (TAIC) | (TAIC) |
| | | | | | | 30 | | | | |
| | | | | | | (ECN1273) | | | | |
| | | | | | | 10 | | | | |
| | | | | | | (TAIC) | | | | |
| Cure acceler- | 6 | 5 | 6 | 6 | 5 | 3 | 1 | 5 | 6 | 6 |
| ating agent | (PH25B) | (PH25B) | (PH25B) | (PH25B) | (2E4MZ) | (PH25B) | (Co) | (Bis S) | (PH25B) | (PH25B) |
| | | | | | | 0.5 | | | | |
| | | | | | | (2E4MZ) | | | | |
| Flame retar- | 20 | 20 | 20 | 20 | | | | | 20 | 20 |
| dant | | | | | | | | | | |
| Inorganic | 50 | | 100 | | | | | 50 | 50 | 50 |
| filler | | | | | | | | | | |
| Methylene | ◯ | ◯ | ◯ | ◯ | ◯ | ◯ | ◯ | ◯ | ◯ | |
| chloride | | | | | | | | | | |
| resistance | | | | | | | | | | |
| Melt viscosity | 9000 | 15000 | 13000 | 6000 | 2000 | 3000 | 3000 | 5000 | 20000 | |
| (poise) | | | | | | | | | | |
| Dielectric | 3.3 | 3.1 | 3.3 | 3.3 | 3.5 | 3.6 | 3.5 | 3.5 | 3.3 | |
| constant | | | | | | | | | | |
| Dielectric | 0.004 | 0.003 | 0.002 | 0.004 | 0.007 | 0.008 | 0.008 | 0.008 | 0.004 | |
| loss tangent | | | | | | | | | | |
| Tg (° C.) | 185 | 185 | 185 | 185 | 170 | 180 | 175 | 180 | 185 | |
| Thermal expansion | 60 | 80 | 50 | 90 | 100 | 90 | 90 | 90 | 60 | |
| coefficent | | | | | | | | | | |
| (from room tem- | | | | | | | | | | |
| perature to Tg) | | | | | | | | | | |
| Soldering heat | ◯ | ◯ | ◯ | ◯ | ◯ | ◯ | ◯ | ◯ | ◯ | |
| resistance | | | | | | | | | | |

Notes for Table 2:
PPE A: Precursory modified polyphenylene ether produced in Synthesis Example 1.
PPE D: Modified polyphenylene ether produced in Exampe 1.
PPE E: Low molecular weight polyphenylene ether produced in Comparative Example 1.
TAIC: Triallyl isocyanurate
AER260: Bisphenol A epoxy resin (AER260, manufactured and sold by Asahi CIBA Co., Ltd., Japan)
ECN1273: Cresol novolak epoxy resin (ECN1273, manufactured and sold by Asahi CIBA Co., Ltd., Japan)
B-10: Cyanate ester resin (B-10, manufactured and sold by Asahi CIBA Co., Ltd., Japan)
B-a: Benzoxazine resin (B-a type, manufactured and sold by SHIKOKU CORPORATION, Japan)
PH25B: Radical initiator (Perhexine 25B, manufactured and sold by Nipppon Oil & Fats Co., Ltd., Japan)
2E4MZ: 2-ethyl-4-methylimidazol (manufactured and sold by SHIKOKU CORPORATION, Japan)
Co: Cobalt naphthenate
Bis S: Bisphenol S
Flame retardant: Bromine-containing flame retardant (Saytex 8010, manufactured and sold by Ethyl Co., Ltd., Japan)
Inorganic filler: Crushed silica (manufactured and sold by TATSUMORI Ltd., Japan)

TABLE 3

| | Ex. 14 | Ex. 15 | Ex. 16 | Ex. 17 | Ex. 18 |
|---|---|---|---|---|---|
| Modified PPE | 100 (F) | 100 (F) | 100 (F) | 100 (F) | 100 (G) |
| Phenolic | 1.5 | 3.0 | 4.5 | 0.8 | 1.5 |
| compound | (BPA) | (BPA) | (BPA) | (XY) | (BPA) |
| Radical | 2.5 | 5 | 7.5 | 3 | 2.5 |
| generator | | | | | |
| Toluene | 550 | 550 | 550 | 550 | 550 |
| Mn | 9000 | 6100 | 5100 | 10700 | 10000 |
| (ratio, relative | (0.53) | (0.36) | (0.30) | (0.63) | (0.55) |
| to precursory PPE) | | | | | |
| Mw | 28000 | 24000 | 22000 | 35000 | 32000 |
| (ratio, relative | (0.78) | (0.67) | (0.61) | (0.97) | (0.84) |
| to precursory PPE) | | | | | |
| Mz | 58000 | 52000 | 47000 | 72000 | 63000 |
| (ratio, relative | (1.07) | (0.96) | (0.87) | (1.33) | (1.11) |
| to precursory PPE) | | | | | |

Notes
Reaction product F: Precursory modified polyphenylene ether produced in Synthesis Example 3. (Mn: 17000, Mw: 36000, Mz: 54000)
Reaction product G: Precursory modified polyphenylene ether produced in Synthesis Example 4. (Mn: 18300, Mw: 38000, Mz: 57000)
BPA: Bisphenol A
XY: 2,6-xylenol
Radical generator: PERROYL TCP (manufactured and sold by Nippon Oil & Fats Co., Ltd., Japan)
Ratio, relative to precursory PPE: (molecular weight of the final PPE)/(molecular weight of the PPE before the reaction)

TABLE 4

| | | | | | | | | | Compara. |
|---|---|---|---|---|---|---|---|---|---|
| | Ex. 19 | Ex. 20 | Ex. 21 | Ex. 22 | Ex. 23 | Ex. 24 | Ex. 25 | Ex. 26 | Ex. 6 |
| Modified PPE | 60 (H) | 70 (H) | 50 (H) | 60 (H) | 30 (H) | 40 (H) | 30 (H) | 45 (H) | 60 (F) |
| Thermocurable resin | 40 | 30 | 50 | 40 | 70 | 30 | 70 | 55 | 40 |
| | (TAIC) | (TAIC) | (TAIC) | (TAIC) | (AER260) | (AER260) | (B-10) | (B-a) | (TAIC) |
| | | | | | | 30 | | | |
| | | | | | | (ECN1273) | | | |
| | | | | | | 10 | | | |
| | | | | | | (TAIC) | | | |
| Cure accelerating | 6 | 5 | 6 | 6 | 5 | 3 | 1 | 5 | 6 |
| agent | (PH25B) | (PH25B) | (PH25B) | (PH25B) | (2E4MZ) | (PH25B) | (Co) | (Bis S) | (PH25B) |
| | | | | | | 0.5 | | | |
| | | | | | | (2E4MZ) | | | |
| Flame retardant | 20 | 20 | 20 | 20 | | | | | 20 |
| Inorganic filler | 50 | | 100 | | | | | 50 | 50 |
| Methylene chloride | ◯ | ◯ | ◯ | ◯ | ◯ | ◯ | ◯ | ◯ | ◯ |
| resistance | | | | | | | | | |
| Melt viscosity | 7000 | 12000 | 10000 | 5000 | 1000 | 2400 | 2500 | 4000 | 20000 |
| (poise) | | | | | | | | | |
| Dielectric constant | 3.3 | 3.1 | 3.4 | 3.3 | 3.6 | 3.6 | 3.5 | 3.5 | 3.4 |
| Dielectric loss tan- | 0.004 | 0.002 | 0.003 | 0.004 | 0.008 | 0.008 | 0.008 | 0.008 | 0.004 |
| gent | | | | | | | | | |
| Tg (° C.) | 185 | 185 | 185 | 185 | 170 | 180 | 175 | 180 | 185 |
| Thermal expansion coef- | 60 | 80 | 50 | 90 | 100 | 90 | 90 | 90 | 60 |
| ficient (from room tem- | | | | | | | | | |
| perature to Tg) | | | | | | | | | |
| soldering heat resis- | ◯ | ◯ | ◯ | ◯ | ◯ | ◯ | ◯ | ◯ | ◯ |
| tance | | | | | | | | | |

Notes for Table 4:
PPE F: Precursory modified polyphenylene ether produced in Synthesis Example 3.
PPE H: Modified polyphenylene ether produced in Example 14.
TAIC: Triallyl isocyanurate
AER260: Bisphenol A epoxy resin (AER260, manufactured and sold by Asahi CIBA Co., Ltd., Japan)
ECN1273: Cresol novolak epoxy resin (ECN1273, manufactured and sold by Asahi CIBA Co., Ltd., Japan)
B-10: Cyanurate ester resin (B-10, manufactured and sold by Asahi CIBA Co., Ltd., Japan)
B-a: Benzoxazine resin (B-a type, manufactured and sold by SHIKOKU CORPORATION, Japan)
PH25B: Radical initiator (Perhexine 25B, manufactured and sold by Nippon Oil & Fats Co., Ltd., Japan)
2E4MZ: 2-ethyl-4-methylimidazol (manufactured and sold by SHIKOKU CORPORATION, Japan)
Co: Cobalt naphthenate
Bis S: Bisphenol S
Flame retardant: Bromine-containing flame retardant (Saytex 8010, manufactured and sold by Ethyl Co., Ltd., Japan)
Inorganic filler: Crushed silica (manufactured and sold by TATSUMORI Ltd., Japan)

### Industrial Applicability

The modified polyphenylene ether of the present invention exhibits excellent melt properties, excellent mechanical properties and excellent film-forming properties, so that the modified polyphenylene ether can be advantageously used in a curable film and a multi-layer circuit board as well as in the build-up method and the like.

1. A modified polyphenylene ether having a number average molecular weight of not smaller than 4,000, which is obtained by reacting a precursory modified polyphenylene ether with a phenolic compound in the presence of a radical generator,
said precursory modified polyphenylene ether comprising a plurality of polyphenylene ether chains each having a terminal phenolic hydroxyl group, said plurality of polyphenylene ether chains collectively having at least one terminal phenolic hydroxyl group modified, wherein not all of the terminal phenolic hydroxyl groups of said plurality of polyphenylene ether chains are modified.

2. The modified polyphenylene ether according to claim 1, wherein said precursory modified polyphenylene ether is a reaction product of a polyphenylene ether with an unsaturated carboxylic acid or an unsaturated carboxylic anhydride.

3. The modified polyphenylene ether according to claim 1, wherein said precursory modified polyphenylene ether is a reaction product of a polyphenylene ether with at least one vinyl compound selected from the group consisting of an acrylic ester and a methacrylic ester, each of said acrylic ester and said methacrylic ester containing a C₉-C₂₂ alkyl group, a C₉-C₂₂ alkenyl group, a C₉-C₂₂ aralkyl group or a C₉-C₂₂ cycloalkyl group.

4. A curable polyphenylene ether resin composition comprising 1 to 99 parts by weight of the modified polyphenylene ether of any one of claims 1 to 3 and 99 to 1 part by weight of a thermocurable resin, the total amount of said modified polyphenylene ether and said thermocurable resin being 100 parts by weight.

5. The curable polyphenylene ether resin composition according to claim 4, wherein said thermocurable resin is at least one member selected from the group consisting of triallyl isocyanurate, triallyl cyanurate, a cyanate ester resin, an epoxy resin and a benzoxazine resin.

6. A curable film comprising the curable polyphenylene ether resin composition of claim 4 or 5.

7. A cured film obtained by curing the curable film of claim 6.

8. A curable structure comprising an insulating layer of the curable film of claim 6 and, disposed thereon, an electroconductive layer of a metallic foil.

9. A cured structure obtained by curing the curable structure of claim 8.

10. A curable composite material comprising a substrate and the curable polyphenylene ether resin composition of claim 4 or 5.

11. A cured composite material obtained by curing the curable composite material of claim 10.

12. A method for producing a modified polyphenylene ether having a number average molecular weight of not smaller than 4,000, which comprises performing a homogeneous reaction of a precursory modified polyphenylene ether with a phenolic compound in the presence of a radical generator,
said precursory modified polyphenylene ether comprising a plurality of polyphenylene ether chains each having a terminal phenolic hydroxyl group, said plurality of polyphenylene ether chains collectively having at least one terminal phenolic hydroxyl group modified, wherein not all of the terminal phenolic hydroxyl groups of said plurality of polyphenylene ether chains are modified.

13. A method for producing a varnish of a curable polyphenylene ether resin composition, which comprises:
performing a homogeneous reaction of a precursory modified polyphenylene ether with a phenolic compound in the presence of a radical generator,
said precursory modified polyphenylene ether comprising a plurality of polyphenylene ether chains each having a terminal phenolic hydroxyl group, said plurality of polyphenylene ether chains collectively having at least one terminal phenolic hydroxyl group modified, wherein not all of the terminal phenolic hydroxyl groups of said plurality of polyphenylene ether chains are modified,
thereby obtaining a reaction mixture containing a modified polyphenylene ether having a number average molecular weight of not smaller than 4,000,
isolating said modified polyphenylene ether from said reaction mixture, and
adding said isolated, modified polyphenylene ether and a thermocurable resin to an organic solvent, followed by mixing, to thereby obtain a curable polyphenylene ether resin composition in a varnish form.

14. A method for producing a varnish of a curable polyphenylene ether resin composition, which comprises:
performing a homogeneous reaction of a precursory modified polyphenylene ether with a phenolic compound in the presence of an organic solvent and a radical generator,
said precursory modified polyphenylene ether comprising a plurality of polyphenylene ether chains each having a terminal phenolic hydroxyl group, said plurality of polyphenylene ether chains collectively having at least one terminal phenolic hydroxyl group modified, wherein not all of the terminal phenolic hydroxyl groups of said plurality of polyphenylene ether chains are modified,
thereby obtaining a reaction mixture containing a modified polyphenylene ether having a number average molecular weight of not smaller than 4,000, and
adding a thermocurable resin to said reaction mixture to obtain a curable polyphenylene ether resin composition in a varnish form.

15. The method according to claim 13 or 14, wherein said curable polyphenylene ether resin composition comprises 1 to 99 parts by weight of said modified polyphenylene ether and 99 to 1 part by weight of said thermocurable resin, the total amount of said modified polyphenylene ether and said thermocurable resin being 100 parts by weight.

16. The method according to claim 15, wherein said thermocurable resin is at least one member selected from the group consisting of triallyl isocyanurate, triallyl cyanurate, a cyanate ester resin, an epoxy resin and a benzoxazine resin.

17. A method for producing a curable film, which comprises applying a varnish obtained by the method of any one of claims 13 to 16 onto a support to obtain a coating, and peeling off said coating from said support to obtain a curable film.
